(19) United States
(12) Patent Application Publication
(10) Pub. No.: US 2011/0091901 A1
(43) Pub. Date: Apr. 21, 2011

(54) Vectors and Methods for Screening Cells for High Expression of Protein of Interest (POI)

(75) Inventors: Philippe Dupraz, Crissier (CH); Michel Kobr, Echandens (CH)

(73) Assignee: MERCK SERONO SA, Coinsins, Vaud (CH)

(21) Appl. No.: 12/893,410

(22) Filed: Sep. 29, 2010

Related U.S. Application Data
(62) Division of application No. 12/064,287, filed on Feb. 20, 2008, now abandoned.
(60) Provisional application No. 60/714,500, filed on Sep. 6, 2005.

(30) Foreign Application Priority Data
Aug. 26, 2005 (EP) 05018634.5

(51) Int. Cl. G01N 33/53; C12Q 1/02; C12P 21/06; C12N 5/10; C12N 15/63

(52) U.S. Cl. 435/7.1; 435/29; 435/69.1; 435/69.7; 435/325; 435/366; 435/320.1

(57) ABSTRACT

This invention refers to industrial production of proteins. More particularly, the invention refers to a fusion protein as a novel chimeric selection marker comprising a peptide conferring resistance to an antibiotic, or a fragment, allelic variant, splice variant or mutein thereof, and at least one sequence comprising SEQ ID NO: 1, 2 or 3, preferably for producing a protein of interest (POI). The inventive chimeric selection marker exhibits: (i) a resistance to an antibiotic; and (ii) a fluorescence activity upon binding of a ligand to the sequence comprising SEQ ID NO: 1, 2 or 3. The invention further refers to nucleic acids encoding the inventive fusion protein and to expression vectors, comprising the inventive fusion protein and additionally the protein of interest (POI). Finally, uses of the inventive chimeric selection marker for screening cells for high expression of a protein of interest (POI) are disclosed.

### CROSS-REFERENCE TO RELATED APPLICATIONS

This application is a divisional of U.S. Ser. No. 12/064,287, filed Feb. 20, 2008, which is the U.S. national stage application of International Patent Application No. PCT/EP2006/065682, filed Aug. 25, 2006, which claims the benefit of U.S. Provisional Patent Application No. 60/714,500, filed Sep. 6, 2005, the disclosures of which are hereby incorporated by reference in their entireties, including all figures, tables and amino acid or nucleic acid sequences.

### FIELD OF THE INVENTION

This invention refers to industrial production of proteins. More particularly, the invention refers to a fusion protein as a novel chimeric selection marker comprising a peptide conferring resistance to an antibiotic, or a fragment, allelic variant, splice variant or mutein thereof, and at least one sequence comprising SEQ ID NO: 1, 2 or 3, preferably for producing a protein of interest (POI). The inventive chimeric selection marker exhibits: (i) a resistance to an antibiotic; and (ii) a fluorescence activity upon binding of a ligand to the sequence comprising SEQ ID NO: 1, 2 or 3. The invention further refers to nucleic acids encoding the inventive fusion protein and to expression vectors, comprising the inventive fusion protein and additionally the protein of interest (POI). Finally, uses of the inventive chimeric selection marker for screening cells for high expression of a protein of interest (POI) are disclosed.

### BACKGROUND

Transfection of DNA into mammalian cells is a common technique, often used to study the effects of transient protein expression or to develop stable cell lines. Such methods allow to study the structure-function relationship of proteins of interest (POI). However, it is difficult to monitor the success of these experiments until the endpoint of reaction is reached. Particularly in the case of transient expression, it is desirable to determine e.g. the transfection efficiency or the expression rate. However, reporter molecules used for the control of the transfection efficiency or the expression rate, e.g. chloramphenicol acetyltransferase or β-galactosidase, typically require cells to be fixed and incubated with an exogeneous substrate, e.g. an heterologous gene. Introducing heterologous genes into animal host cells and screening for expression of the added genes is a lengthy and complicated process. Some major problems to be overcome are e.g.: (i) the construction of large expression vectors; (ii) the transfection and selection of clones with stable long-term expression, eventually in the absence of selective pressure; and (iii) screening for high expression rates of the heterologous protein of interest.

Selection of the clones, having integrated the gene of interest and/or highly expressing the protein of interest, is typically performed using one marker system which allows a skilled person to pre-select clones by means of a simple selection system.

One typical approach is the use of selection markers conferring resistance to selective pressure. Most of these selection markers confer resistance to an antibiotic such as, e.g. neomycin, kanamycin, hygromycin, gentamycin, chloramphenicol, puromycin, zeocin or bleomycin. When generating cell clones expressing a gene of interest from expression vectors, host cells are typically transfected with a plasmid DNA vector encoding both a protein of interest and selection marker as mentioned above on the same vector. However, the plasmid capacity to incorporate gene sequences is normally limited and, accordingly, the selection marker has to be expressed by a second plasmid, which is co-transfected with the plasmid comprising the gene of interest.

Stable transfection typically leads to random integration of the expression vector into the genome of the host cell. Use of selective pressure, e.g. by administering an antibiotic to the medium, eliminates all cells that did not integrate the vector containing the selection marker providing resistance to the respective antibiotic or selective pressure in general. If the selection marker is located on the same vector as the gene of interest, or alternatively, if the selection marker is located on a second vector being co-transfected with the vector comprising the gene of interest, the cells will express both the selection marker and the gene of interest. It is frequently observed, however, that the expression level of the gene of interest is highly variable depending on the integration site.

Furthermore, when removing selective pressure from the system, it is frequently observed that expression becomes unstable or even vanishes. Only a small number of initial transfectants are thus providing high and stable long-term expression and it is extremely tedious to identify these clones in large candidate populations. Thus, it would be advantageous in these systems to cultivate candidate clones in the absence of selective pressure in a first step, following an initial period of selection for stable transfection, in order to obtain a large candidate population. Subsequently, in a second step, candidate clones may be screened for expression of a gene of interest. However, then no selection can be carried out upon applying selective pressure as known for prior art methods.

In another approach, screening for clones highly-expressing the protein of interest can be carried out by methods directly revealing the presence of high protein amounts. Typically, immunologic methods, such as ELISA or immunohistochemical staining, are applied to detect the integrated product either intracellularly or in cell culture supernatants. These methods are often tedious, expensive, time-consuming, and typically not amenable to High-Throughput-Screening (HTS)-Assays. It is to be noted that, in addition, an antibody specific for the expressed protein must be available in order to enable detection of the expressed protein.

Attempts to quantify the protein amounts by Fluorescence-Activated Cell Sorting (FACS) have also been made, but only with a limited success, especially in the case of secreted proteins (see e.g. Borth et al. (2000); Biotechnol. Bioeng. 71, 266-273). The FACS technology is based on the step of tagging subpopulations of cells with a detectable marker and sorting preferred cells by means of a signal excited by this marker.

Numerous easily detectable markers are available in the art. They usually correspond to enzymes which act on chromogenic or luminogenic substrates such as, e.g. β-glucuronidase, chloramphenicol acetyltransferase, nopaline synthase, β-galactosidase, luciferase and secreted alkaline phosphatase (SEAP). Fluorescent proteins such as, e.g. Green Fluorescent Protein (GFP) or the synthetic peptide as described by Griffin et al. (“Specific covalent labeling of recombinant protein molecules inside live cells” Science, 1998, Jul. 10; 281 (5374): 269-72) may be used as detectable markers in FACS. The activity of all these proteins and peptides can be measured by standard assays that may be established in High-Throughput-Screening (HTS)-formats.

One general approach for the screening of high expression rates of the protein of interest refers to the use of two detectable selection markers, each having selection properties. Such a selection marker system, having two separate markers, makes use of a detectable marker and an additional marker, expressed from the same vector as the gene of interest (see e.g. Chesnut et al. (1996); J. Immunol. Methods 193, 17-27). The underlying idea of this concept of using such a detectable selection marker system is to establish a correlation between the expression of the gene of interest and the additional marker due to co-expression of the two separate genes on the same vector.

The drawback of this approach is the use of yet another expression cassette for the additional selection marker. This renders the expression vector rather bulky by hosting expression units comprising a promoter, a cDNA and polyadenylation signals for at least three proteins (i.e., the gene of interest, the selection marker and an additional marker). For multi-chain proteins the situation becomes even more complex. Alternatively, individual plasmid vectors expressing the three genes, which encode (a) the protein of interest, (b) the selection marker and (c) the additional selection marker, respectively, could be co-transfected. However, it is likely that the vectors will be either integrated at different loci, or exhibit varying and uncorrelated and additionally very low expression rates. Moreover, proteins expressed with very low expression rates may be inactive or misfolded due to ineffective or defective translation. As a consequence, in such constructs, the protein of interest should not exceed a defined molecular weight (which, however, depends on the expression system used) when using bulky detectable markers in order to allow effective translation to at least some extent. Nevertheless, this significantly lowers applicability of the above method.

Another approach to overcome the above limitations consists in the use of a chimeric marker that combines the functional properties of a selection marker and of a detectable marker. Some chimeric markers have been described in the art.

For example, Bennett et al. (1998, Biotechniques 24, 478-482) discloses the GFP-Zeo^{R} marker, which confers resistance to the Zeocin antibiotic, the expression of which can be monitored by fluorescence microscopy. This article suggests that the GFP-Zeo^{R} marker may be useful for screening for expression of a protein of interest. However, there are no experimental data actually demonstrating that expression of the protein of interest is indeed correlated with expression of the GFP-Zeo^{R} marker.

US 2004/0115704 discloses a puro-GFP chimeric marker as well as its use for measuring the activity of a transcriptional control element. US 2004/0115704 neither teaches nor suggests the use of such a marker for screening cells for expression of a protein of interest.

WO 2006/058900 discloses a fusion protein comprising a luciferase and the puromycin N-acetyl transferase, particularly the use of luciferases derived from a firefly such as, e.g., photinus pyralis, Luciola cruciata, Luciola lateralis or Photuris pennsylvanica, from Renilla reniformis (sea pansy) or from Vargula hilgendorfii (sea firefly) fused in frame with puromycin N-acetyl transferase. This fusion protein allows to combine the functional properties of a selection marker (puromycin) and a detectable marker (luciferase activity).

WO 01/53325 relates to methods of using the synthetic peptide described by Griffin et al. (1998), further referred to as Lumio-Tag. Specifically, WO 01/53325 teaches methods for affinity purification of a protein of interest using a modified fluorescent compounds immobilized to a solid support. In such methods, the protein of interest is fused to a Lumio-Tag. WO 01/53325 further teaches DNA constructs which includes (i) the protein of interest fused to a Lumio-Tag; and (ii) a selectable marker, said selectable marker corresponding to a gene conferring resistance to an antibiotic. However, on these DNA constructs the gene encoding the protein of interest fused to the Lumio-Tag is a different gene from the gene conferring resistance to an antibiotic. In other words, WO 01/53325 does not disclose any chimeric marker comprising the Lumio-Tag, but only chimeric protein of interests. In addition, the DNA constructs of WO 01/53325 are used for protein purification and not for screening for clones highly-expressing a protein of interest.

Thus, the problems resulting from the use of state-of-the-art markers are not yet solved. There still exists a need of providing efficient chimeric markers. The provision of a novel, alternative and powerful chimeric marker would be extremely useful in the field of industrial production of therapeutic proteins and for screening for high-expressing clones.

### DESCRIPTION

Therefore, the object underlying the present invention is to provide a chimeric marker system allowing both to select cells and to monitor expression of a protein of interest (POI), without being limited by a strict size limitation for the proteins of interest.

The above object is solved by an inventive chimeric selection marker provided as a fusion protein comprising a peptide conferring resistance to an antibiotic, or a fragment, allelic variant, splice variant or mutein thereof and at least one sequence comprising SEQ ID NO: 1, 2 or 3, wherein the inventive chimeric selection marker exhibits: (i) a resistance to said antibiotic; and (ii) a fluorescence activity upon binding of a ligand to said sequence comprising SEQ ID NO: 1, 2 or 3. If the inventive chimeric selection marker is incorporated into the cell, the cell is characterized by cell survival upon addition of the corresponding antibiotic and emits fluorescent light, if a suitable ligand is added.

The inventive fusion protein comprises as a first component a peptide conferring resistance to an antibiotic. This antibiotic is preferably selected from neomycin, kanamycin, neomycin-kanamycin, hygromycin, gentamycin, chloramphenicol, puromycin, zeocin or bleomycin, respectively.

The peptides used as a first component and conferring resistance to these antibiotics are preferably encoded by a corresponding resistance gene. Preferably, the resistance gene is selected from the resistance genes for the above mentioned antibiotics, e.g. the gene encoding neomycin phosphotransferase type II, the gene encoding kanamycin phosphotransferase type II, the gene encoding neomycin-kanamycin phosphotransferase type II, the gene encoding hygromycin phosphotransferase, the gene encoding gentamycin acetyl transferase, the gene encoding chloramphenicol acetyltransferase, the gene encoding puromycin N-acetyl transferase (pac), the gene encoding the zeocin resistance protein or the gene encoding the bleomycin resistance protein, or a fragment, allelic variant, splice variant or mutein thereof. The (biological) activity of peptides encoded by these resistance genes, is their capability of conferring resistance to the above mentioned antibiotics.

More preferably, the inventive fusion protein comprises as a first component a peptide conferring a resistance for an antibiotic selected from:
(i) a puromycin N-acetyltransferase according to SEQ ID NO: 5 as encoded by the puromycin N-acetyltransferase resistance gene according to SEQ ID NO: 4;
(ii) a neomycin phosphotransferase type II according to SEQ ID NO: 7 as encoded by the neomycin resistance gene according to SEQ ID NO: 6;
(iii) a kanamycin phosphotransferase type II according to SEQ ID NO: 9 as encoded by the kanamycin resistance gene according to SEQ ID NO: 8;
(iv) a neomycin-kanamycin phosphotransferase type II according to SEQ ID NO: 11 as encoded by the neomycin-kanamycin resistance gene according to SEQ ID NO: 10;
(v) a hygromycin phosphotransferase according to SEQ ID NO: 13 as encoded by the hygromycin resistance gene according to SEQ ID NO: 12;
(vi) a gentamycin acetyltransferase according to SEQ ID NO: 15 as encoded by the gentamycin resistance gene according to SEQ ID NO: 14;
(vii) a chloramphenicol acetyltransferase according to SEQ ID NO: 17 as encoded by the chloramphenicol resistance gene according to SEQ ID NO: 16;
(viii) a zeocin resistance protein according to SEQ ID NO: 19 as encoded by the zeocin resistance gene according to SEQ ID NO: 18; and/or
(ix) a bleomycin resistance protein according to SEQ ID NO: 21 as encoded by the bleomycin resistance gene according to SEQ ID NO: 20.

More preferably, the inventive fusion protein comprises as a first component a puromycin-N-acetyltransferase. As mentioned above, the (biological) activity of puromycin-N-acetyltransferase according to the present invention is its capability of conferring resistance to puromycin. Puromycin (puromycin dihydrochloride[3′(α-Amino-p-methoxyhydrocinnamamido)-3′-deoxy-N,N-dimethyladenosine.2HCl], C₂₂H₂₉N₇O₅.2HCl, MW.: 544.43 (Sambrook, J., Fritsch, E. F. & Maniatis, T.; Molecular Cloning: A Laboratory

Manual, 3rd Edition. Cold Spring Harbor Laboratory Press, Cold Spring Harbor, N.Y.)) is an aminonucleoside antibiotic from Streptomyces alboniger. It is an analogon to aminoacyl-tRNA and inhibits the protein synthesis by termination of the peptidyl transfer at the ribosomes in prokaryotes and eukaryotes. The antibiotic inhibits the growth of gram positive bacteria and different animal cells. Fungi and gram negative bacterias are resistant, since puromycin cannot pass the cell wall. Stock concentrations of puromycin are typically 5-50 mg/ml in dH₂O, store at −20° C., the working concentrations are typically 1-30 μg/ml (mammalian cell).

Even more preferably, the puromycin N-acetyl transferase (pac) to be used as a first component of the inventive fusion protein is a native sequence from microorganisms, preferably derived from a Streptomyces species such as Streptomyces alboniger or Streptomyces coelicolor. Preferably, the puromycin N-acetyl transferase (pac) of the inventive fusion protein is a native full-length sequence, more preferably, a native full-length sequence derived from Streptomyces alboniger pac. In a more preferred embodiment, the puromycin N-acetyl transferase (pac) of the inventive fusion protein comprises a peptide sequence according to SEQ ID NO: 5 or a peptide sequence encoded by SEQ ID NO: 4. Even more preferably, the puromycin N-acetyl transferase (pac) of the inventive fusion protein comprises amino acids 2 to 199 of SEQ ID NO: 5 or a peptide as encoded by nucleotides 3 to 597 according to SEQ ID NO: 4. Native puromycin N-acetyltransferases also encompass all naturally occurring splice variants. A “splice variant” of the puromycin N-acetyl transferase (pac) as defined above shall be understood as a puromycin N-acetyl transferase obtained by different, non-canonical splicing of the unspliced peptide of native puromycin N-acetyl transferase (pac) as defined above. More preferably, such a splice variant of the puromycin N-acetyl transferase (pac) still exhibits puromycin N-acetyl transferase (pac)-activity.

In one alternative embodiment, the inventive fusion protein comprises as a first component a fragment of a peptide conferring a resistance to an antibiotic as defined above. According to the present invention a fragment of an such a peptide is defined as a sequence having at least 50%, more preferably at least 60%, and still more preferably at least 70%, 80%, 85%, 90%, 95%, 96%, 97%, 98% or 99% sequence identity with its corresponding native peptide, wherein these fragments still confer resistance to their corresponding antibiotics (functionally active).

Alternatively or additionally, the first component of the inventive fusion protein (or the inventive fusion protein or a protein of interest as defined below) may correspond to a biologically active fragment of at least 50, 100 or 150 amino acids of its native full-length form, i.e. the native full-length form of the peptide conferring resistance to an antibiotic as defined above (or the inventive fusion protein or a protein of interest as defined below). Importantly, this fragment is still biologically active and confers resistance to an antibiotic as defined above. The (biological) activity of the first component can for example be measured by routine methods as known to a skilled person.

In still another embodiment, the first component of the inventive fusion protein comprises allelic variants of a peptide conferring resistance to an antibiotic as defined above. According to the present invention an “allelic variant” shall be understood as an alteration in the native sequence of the native form of the first component as defined above, wherein the altered sequence still confers resistance to the corresponding antibiotic. More preferably, an allelic variant of the first component as defined above has at least 50%, more preferably at least 60%, and still more preferably at least 70%, 80%, 85%, 90%, 95%, 96%, 97%, 98% or 99% sequence identity with the native form of the first component, more preferably with a sequence as defined above, e.g. SEQ ID NO: 5, more preferably with amino acids 2 to 199 of SEQ ID NO: 5, or with a sequence according to SEQ ID NOs: 7, 9, 11, 13, 15, 17, 19 or 21. The allelic variants of the first component, i.e. allelic variants of a peptide conferring resistance to an antibiotic, still confer resistance to their corresponding antibiotic, i.e. neomycin, kanamycin, neomycin-kanamycin, hygromycin, gentamycin, chloramphenicol, puromycin, zeocin or bleomycin.

The (biological) activity of the first component, i.e. conferring resistance to its corresponding antibiotic, may also be conferred by a mutein of the first component. As used herein, the term “mutein” refers to an analog of a naturally occurring polypeptide, e.g. an analog of the native form of the first component as defined above, in particular an analog of the sequences 5, 7, 9, 11, 13, 15, 17, 19 and 21 (or the inventive fusion protein or a protein of interest as defined below), in which one or more of the amino acid residues of the naturally occurring polypeptide are replaced by different amino acid residues, or are deleted, or one or more amino acid residues are added to the naturally occurring sequence of the polypeptide, without considerably lowering the activity of the resulting products as compared with the naturally occurring polypeptide. These muteins are prepared by known synthesis and/or by site-directed mutagenesis techniques, or any other known technique suitable therefore. Muteins of the first component as defined above (or of the inventive fusion protein or of a protein of interest as defined below) that can be used in accordance with the present invention, or nucleic acids encoding these muteins, preferably include a finite set of substantially corresponding sequences as substitution polypeptides or polynucleotides which can be routinely obtained by one of ordinary skill in the art, without undue experimentation, based on the teachings and guidance presented herein.

Muteins of the first component as defined above (or the inventive fusion protein or of a protein of interest as defined below) in accordance with the present invention preferably include proteins encoded by a nucleic acid, such as DNA or RNA, which hybridizes to DNA or RNA, which encode the (native form of the) first component as defined above, under moderately or highly stringent conditions. The term “stringent conditions” refers to hybridization and subsequent washing conditions, which those of ordinary skill in the art conventionally refer to as “stringent”. See Ausubel et al., Current Protocols in Molecular Biology, supra, Interscience, N.Y., 6.3 and 6.4 (1987, 1992), and Sambrook et al. (Sambrook, J. C., Fritsch, E. F., and Maniatis, T. (2001) Molecular Cloning: A Laboratory Manual, Cold Spring Harbor Laboratory Press, Cold Spring Harbor, N.Y.).

Without limitation, examples of stringent conditions include washing conditions at 12-20° C. below the calculated Tₘ of the hybrid under study in, e.g., 2×SSC and 0.5% SDS for 5 minutes, 2×SSC and 0.1% SDS for 15 minutes; 0.1×SSC and 0.5% SDS at 37° C. for 30-60 minutes and then, 0.1×SSC and 0.5% SDS at 68° C. for 30-60 minutes. Those of ordinary skill in this art understand that stringency conditions also depend on the length of the DNA sequences, oligonucleotide probes (such as 10-40 bases) or mixed oligonucleotide probes. If mixed probes are used, it is preferable to use tetramethyl ammonium chloride (TMAC) instead of SSC.

Muteins of the first component as defined above (or of the inventive fusion protein or of a protein of interest as defined below) include polypeptides having an amino acid sequence being at least 50% identical, more preferably at least 60% identical, and still more preferably at least 70%, 80%, 90%, 95%, 96%, 97%, 98% or 99% identical to their native form, e.g. the native form of the first component, wherein these muteins of the first component still confer resistance to an antibiotic as defined above.

A polypeptide having an amino acid sequence being at least, for example, 95% “identical” to a query amino acid sequence of the present invention, is intended to mean that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence of at least 95% identity to a query amino acid sequence, up to 5% (5 of 100) of the amino acid residues in the subject sequence may be inserted, deleted, or substituted with another amino acid.

For sequences without exact correspondence, a “% identity” of a first sequence may be determined with respect to a second sequence. In general, these two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or similar length, or over shorter, defined lengths (so-called local alignment), that is more suitable for sequences of unequal length.

Methods for comparing the identity and homology of two or more sequences are well known in the art. Thus for instance, programs available in the Wisconsin Sequence Analysis Package, version 9.1 (Devereux et al., 1984, Nucleic Acids Res. 12, 387-395.), for example the programs BESTFIT and GAP, may be used to determine the % identity between two polynucleotides and the % identity and the % homology between two polypeptide sequences. BESTFIT uses the “local homology” algorithm of (Smith and Waterman (1981), J. Mol. Biol. 147, 195-197.) and finds the best single region of similarity between two sequences. Other programs for determining identity and/or similarity between sequences are also known in the art, for instance the BLAST family of programs (Altschul et al., 1990, J. Mol. Biol. 215, 403-410), accessible through the home page of the NCBI at world wide web site ncbi.nlm.nih.gov) and FASTA (Pearson (1990), Methods Enzymol. 183, 63-98; Pearson and Lipman (1988), Proc. Natl. Acad. Sci. U.S.A 85, 2444-2448.).

Preferred changes for muteins in accordance with a fusion protein of the present invention are “conservative” substitutions. Conservative amino acid substitutions of the first component as defined above (or of the inventive fusion protein or of a protein of interest as defined below), may include synonymous amino acids within a group which have sufficiently similar physicochemical properties, so that a substitution between members of the group will preserve the biological function of the molecule (see e.g. Grantham, R. (1974), Science 185, 862-864). It is evident to the skilled person that amino acids may also be inserted and/or deleted in the (above-)defined sequences without altering their function, particularly if the insertions and/or deletions only involve a few amino acids, e.g. less than under thirty, and preferably less than ten, and do not remove or displace amino acids which are critical to functional activity, e.g. cysteine residues.

Preferably, synonymous amino acids, which are classified into the same groups and are typically exchangeable are defined in Table I. More preferably, the synonymous amino acids are defined in Table II, and even more preferably in Table III.

TABLE I
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE II
More Preferred Groups of Synonymous Amino Acids

| Amino Acid | Synonymous Group |
|---|---|
| Ser | Ser |
| Arg | His, Lys, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Lys | Lys, Arg |
| Asp | Asp, Asn |
| Glu | Glu, Gln |
| Met | Met, Phe, Ile, Val, Leu |
| Trp | Trp |

TABLE III
Most Preferred Groups of Synonymous Amino Acids

| Amino Acid | Synonymous Group |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Lys | Lys |
| Asp | Asp |
| Glu | Glu |
| Met | Met, Ile, Leu |
| Trp | Met |

Examples of production of amino acid substitutions in proteins which can be used for obtaining muteins of the first component as defined above (or the inventive fusion protein or of a protein of interest as defined below) for use in the present invention include any known methods, such as presented in U.S. Pat. Nos. 4,959,314, 4,588,585 and 4,737,462, to Mark et al; U.S. Pat. No. 5,116,943 to Koths et al., U.S. Pat. No. 4,965,195 to Namen et al; U.S. Pat. No. 4,879,111 to Chong et al; and U.S. Pat. No. 5,017,691 to Lee et al; and lysine substituted proteins presented in U.S. Pat. No. 4,904,584 (Shaw et al) or as described in Sambrook et al. 2001, Molecular Cloning: A Laboratory Manual, Cold Spring Harbor, N.Y.

Preferably, a mutein of the present invention exhibits substantially the same biological activity as the naturally occurring polypeptide to which it corresponds.

As a second component the inventive fusion protein comprises at least one core sequence according to SEQ ID NO: 1 (Cys Cys Xaa Xaa Cys Cys), having a set of four cysteines at amino acid positions 1, 2, 5 and 6. The amino acids at positions 3 and 4 of SEQ ID NO: 1 may comprise any amino acid, selected from naturally occurring amino acids alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine, or from non-naturally occurring variants thereof, e.g. selenocysteine. More preferably, the amino acids at positions 3 and 4 of SEQ ID NO: 1 comprise a proline or a glycine (SEQ ID NO: 2). The inventive fusion protein may thus comprise as a second component at least one sequence comprising SEQ ID NO: 2. Even more preferably, in SEQ ID NO: 2 a proline is positioned at amino acid position 3 and a glycine is positioned at amino acid position 4. Additionally, any of SEQ ID NOs: 1 and 2 may comprise further amino acids at their N- and/or C-terminus, preferably selected from glycine. An exemplary preferred sequence, present at least once in the inventive fusion protein, is represented by SEQ ID NO: 3.

The second component as contained in the inventive fusion protein, preferably comprises a length of 6 to 50 amino acids, more preferably of 6 to 30 amino acids and even more preferably of 6 to 20 amino acids.

The fusion protein containing a peptide conferring resistance to an antibiotic as defined above, or the fragment, allelic variant, splice variant or mutein thereof, and at least one sequence comprising SEQ ID NO: 1, 2 or 3, is capable of binding to a ligand of the sequence comprising SEQ ID NO: 1, 2 or 3.

A “ligand” in the context of the present invention is preferably a compound, capable of binding to a sequence comprising SEQ ID NO: 1, 2 or 3. Preferably, such a ligand has fluorescent properties. Even more preferably, such a ligand is a fluorescein or a derivative therefrom, and most preferably, the ligand is a membrane permeable biarsenical fluorescein derivative, e.g. the membrane-permeable fluorescein derivative 4′,5′-bis(1,3,2-dithioarsolan-2-yl)fluorescein, or any derivative thereof exhibiting the same binding and fluorescence properties.

The ligand itself is non-fluorescent in its unbound state, but becomes fluorescent upon binding to SEQ ID NO: 1, 2 or 3. It is to be noted, that SEQ ID NO: 1 represents the generic core sequence the ligand requires for binding. However, the core sequence of SEQ ID NO: 1 may be amenable to various specific variants, which are covered by the core sequence as disclosed above. The fluorescence of the ligand in its bound state may be detected using any known fluorescence detection method being suitable for detecting fluorescence signals. Preferred methods include specific generation of fluorescence signals, i.e. exciting fluorescence of the ligand with a defined wavelength, and detecting the generated fluorescence signals subsequently. Simultaneous or time-staggered generation and detection of fluorescence signals of the ligand is encompassed by this invention as well. Preferably, the fluorescence detection is carried out with a laser-induced fluorescence detection (LIF), a laser-induced time-staggered fluorescence detection (LI2F), a Fluorescence Lifetime Imaging Microscopy (FLIM), spectrophotometry, flow cytometry, white fluid fluorescence spectroscopy, or Fluorescence-Activated Cell Sorting (FACS).

Fusing as the first component a peptide conferring resistance to an antibiotic as defined above to at least one sequence according to SEQ ID NO: 1, 2 or 3, to 2, 3 or even more sequences according to SEQ ID NO: 1, 2 or 3, may lead to a fusion protein, which exhibits a stronger fluorescence signal upon binding to the ligand than a fusion protein carrying just one sequence according to SEQ ID NO: 1, 2 or 3. A tagging of more than one of the above-defined ligand binding sequences may be used e.g. for increasing the signal/noise rate, if low fluorescence signals are to be expected, e.g. if other fluorescent components are also present in the probe.

If the first component of the inventive fusion protein or a variant thereof is fused to just one ligand binding sequence comprising SEQ ID NO: 1, 2 or 3 as second component of the inventive fusion protein, the 3′ terminus of the first component, or a fragment, allelic variant, splice variant or mutein thereof, may be linked to the 5′ terminus of a ligand binding sequence comprising SEQ ID NO: 1, 2 or 3, or, preferably, the 3′ terminus of ligand binding sequence comprising SEQ ID NO: 1, 2 or 3 may be fused to the 5′ terminus of the first component or a fragment, allelic variant, splice variant or mutein thereof.

Alternatively, if a first component as defined above or a variant thereof and more than one ligand binding sequence comprising SEQ ID NO: 1, 2 or 3 are contained in the inventive fusion protein, the ligand binding sequence comprising SEQ ID NO: 1, 2 or 3 may be positioned blockwise at the 3′ terminus of the first component, or a fragment, allelic variant, splice variant or mutein thereof, via the 5′ terminus of a ligand binding sequence comprising SEQ ID NO: 1, 2 or 3, and vice versa. In another alternative, two or more ligand binding sequences comprising SEQ ID NO: 1, 2 or 3 may be present at either terminus of the sequence of the first component.

The inventive fusion protein may contain a linker, which spatially separates its afore disclosed first and second component(s). Alternatively (or additionally), such a linker may be used to spatially separate the ligand binding sequences comprising SEQ ID NO: 1, 2 or 3, if a plurality of them is present in the inventive fusion protein. Typically, such a linker is an oligo- or polypeptide. Preferably, the linker has a length of 1-20 amino acids, more preferably a length of 1 to 10 amino acids and most preferably a length of 1 to 5 amino acids. Advantageously, the fusion according to the present invention comprises a linker without secondary structure forming properties, i.e. without an 60 -helix or a β-sheet structure forming tendency. More preferably, the linker is composed of at least 50% of glycin and/or proline residues. Most preferably, the linker is exclusively composed of glycin residues.

The inventive fusion protein or rather its components as defined above (or the protein of interest as defined below), may additionally be labelled for further detection. Such a label is preferably selected from the group of labels comprising:
(i) radioactive labels, i.e. radioactive phosphorylation or a radioactive label with sulphur, hydrogen, carbon, nitrogen, etc.
(ii) coloured dyes (e.g. digoxygenin, etc.)
(iii) fluorescent groups (e.g. fluorescein, etc.)
(iv) chemoluminescent groups,
(v) groups for immobilisation on a solid phase (e.g. His-tag, biotin, strep-tag, flag-tag, antibodies, anti gene, etc.) and
(vi) a combination of labels of two or more of the labels mentioned under (i) to (v).

In a particularly preferred embodiment, the inventive fusion protein comprises the sequence according to SEQ ID NO: 23 or is encoded by the sequence according to SEQ ID NO: 22.

A second aspect of the present invention refers to nucleic acids, encoding the fusion protein as defined above. An inventive nucleic acid encoding the inventive fusion protein may comprise mRNA, RNA, genomic DNA, subgenomic DNA, cDNA, synthetic DNA, and/or combinations thereof. An inventive nucleic acid also includes any nucleic sequence variant encoding the desired amino acid sequence of an inventive fusion protein (due to degeneration of the genetic code). E.g. these alternative nucleic acid sequences may lead to an improved expression of the encoded fusion protein in a selected host organism. Tables for appropriately adjusting a nucleic acid sequence are known to a skilled person. Preparation and purification of such nucleic acids and/or derivatives are usually carried out by standard procedures (see Sambrook et al. 2001, Molecular Cloning: A Laboratory Manual, Cold Spring Harbor, N.Y.). Preferably, said nucleic acid encodes a fusion protein comprising SEQ ID NO: 23. Most preferably, said nucleic acid comprises SEQ ID NO: 22.

A third aspect of the present invention refers to an (expression) vector. The term “vector” is used herein to designate either circular or linear DNA or RNA, which is either double-stranded or single-stranded, and which comprises at least one inventive nucleic acid to be transferred into a cell host or into a unicellular or multicellular host organism. The inventive vector comprises an inventive nucleic acid encoding the inventive fusion protein as defined above and a nucleic acid encoding a protein of interest (POI) or a mutein thereof.

A protein of interest according to the present invention may be any polypeptide the production of which is desired. The protein of interest may be applied in the field of pharmaceutics, agribusiness or furniture for research laboratories. Preferred proteins of interests find use in the field of pharmaceutics. For example, the protein of interest may be, e.g., a naturally secreted protein, a cytoplasmic protein, a transmembrane protein, or a human or a humanized antibody. When the protein of interest is a cytoplasmic or a transmembrane protein, the protein has preferably been altered such as to become soluble. Such an alteration may be carried out by any method known to a skilled person. Preferably, such an alteration is carried out e.g. by increasing the number of codons encoding hydrophilic amino acids in the coding nucleic acid sequence, e.g. by (conservatively) substituting and/or deleting nucleotides of codons encoding lipophilic and/or amphiphilic amino acids. Substitutions in the encoding nucleic acid preferably lead to amino acid substitutions as indicated in any of Tables I to III.

The polypeptide of interest may be of any origin. Preferred polypeptides of interest are of human origin and are selected e.g. from (poly)peptide hormones, cytokines, proteins involved in the blood clotting system, growth factors and factors involved in hematopoiesis.

Preferably, the protein of interest is selected from the group consisting of chorionic gonadotropin, follicle-stimulating hormone, lutropin-choriogonadotropic hormone, thyroid stimulating hormone, human growth hormone, interferons (e.g., interferon beta-la, interferon beta-1b), interferon receptors (e.g., interferon gamma receptor), TNF receptors p55 and p75, interleukins (e.g., interleukin-2, interleukin-11), interleukin binding proteins (e.g., interleukin-18 binding protein), anti-CD11a antibodies, erythropoietin, granulocyte colony stimulating factor, granulocyte-macrophage colony-stimulating factor, pituitary peptide hormones, menopausal gonadotropin, insulin-like growth factors (e.g., somatomedin-C), keratinocyte growth factor, glial cell line-derived neurotrophic factor, thrombomodulin, basic fibroblast growth factor, insulin, Factor VII, Factor VIII, Factor IX, somatropin, bone morphogenetic protein-2, protein-3, protein-4, protein-5, protein-6, protein-7, protein-8, protein-9, protein-10, platelet-derived growth factor, hirudin, erythropoietin, recombinant LFA-3/IgG1 fusion protein, glucocerebrosidase, and muteins, fragments, soluble forms, functional derivatives, fusion proteins thereof, wherein a “mutein” of a protein of interest according to the present invention is as defined above in the general definition for “muteins”.

In a further preferred embodiment, the protein of interest may be labeled for further detection using any of the labels as defined above. Methods for introducing such a label into the protein of interest are known to a skilled person and are described e.g. in Sambrook, J. C., Fritsch, E. F., and Maniatis, T. (2001) Molecular Cloning: A Laboratory Manual, Cold Spring Harbor Laboratory Press, Cold Spring Harbor, N.Y.

Preferably, the inventive vector is an expression vector. An “expression vector” according to the present invention preferably comprises a vector as defined above and additionally appropriate elements as expression support including various regulatory elements, such as enhancers/promoters from viral, bacterial, plant, mammalian, and other eukaryotic sources that drive expression of the inserted polynucleotide in host cells, such as insulators, boundary elements, LCRs (e.g. described by Blackwood and Kadonaga (1998), Science 281, 61-63) or matrix/scaffold attachment regions (e.g. described by Li, Harju and Peterson, (1999), Trends Genet. 15, 403-408).

The term “promoter” as used herein refers to a region of DNA that functions to control the transcription of one or more DNA sequences, and that is structurally identified by the presence of a binding site for DNA-dependent RNA-polymerase and of other DNA sequences, which interact to regulate promoter function. A functional expression promoting fragment of a promoter is a shortened or truncated promoter sequence retaining the activity as a promoter. Promoter activity may be measured by any assay known in the art, e.g. by a reporter assay using luciferase as reporter gene (Wood, de Wet, Dewji, and DeLuca, (1984), Biochem Biophys. Res. Commun. 124, 592-596; Seliger and McElroy, (1960), Arch. Biochem. Biophys. 88, 136-141) or commercially available from Promega®).

In a preferred embodiment, the inventive expression vector comprises at least one promoter of the murine CMV immediate early region. The promoter may for example be the promoter of the mCMV IE1 gene (the “IE1 promoter”), which is known from, e.g. WO 87/03905. The promoter may also be the promoter of the mCMV IE2 gene (the “IE2 promoter”), the mCMV IE2 gene itself being known from, e.g., Messerle, Keil, and Koszinowski. 1991, J. Virol. 65, 1638-1643. The IE2 promoter and the IE2 enhancer regions are described in details in PCT/EP2004/050280.

An “enhancer region” as used in the inventive expression vector, typically refers to a region of DNA that functions to increase the transcription of one or more genes. More specifically, the term “enhancer”, as used herein, is a DNA regulatory element that enhances, augments, improves, or ameliorates expression of a gene irrespective of its location and orientation vis-à-vis the gene to be expressed, and may be enhancing, augmenting, improving, or ameliorating expression of more than one promoter.

Additionally, the inventive expression vector may comprise an amplification marker. This amplification marker may be selected from the group consisting of, e.g. adenosine deaminase (ADA), dihydrofolate reductase (DHFR), multiple drug resistance gene (MDR), ornithine decarboxylase (ODC) and N-(phosphonacetyl)-L-aspartate resistance (CAD).

Amplification of the gene encoding the above defined proteins, i.e. the protein of interest (POI) and/or the inventive fusion protein, allows to increase the expression level of these proteins upon integration of the vector in a cell (Kaufman et al. (1985), Mol. Cell Biol. 5, 1750-1759).

According to one embodiment, the inventive expression vector comprises one promoter or a promoter assembly, wherein this promoter or promoter assembly drives the expression of both the protein of interest (POI) or a mutein thereof, and the inventive fusion protein. Therefore, the protein of interest and the inventive fusion protein are preferably contained “in frame” in one expression cassette in the inventive expression vector, wherein the coding regions of both are separated by an internal ribosomal entry site (IRES), thus forming a bicistronic nucleic acid sequence in the inventive vector. Such a (internal ribosomal entry site) sequence allows the ribosomal machinery to initiate translation from a secondary site within a single transcript and thus to express both the protein of interest and the inventive fusion protein as two separate proteins, when using just one promoter/promoter assembly. This embodiment ensures an optimal correlation between expression of the inventive fusion protein and expression of the POI. Such correlation is essential, when using the inventive fusion protein for screening cells for high expression of a POI.

Alternatively, the inventive expression vector may comprise at least two promoters or promoter assemblies, wherein one of these promoters drives the expression of the inventive fusion protein, and the other one drives the expression of the protein of interest (POI). In this embodiment, the expression vector preferably carries two expression cassettes, the first carrying the inventive fusion protein and the second one the protein of interest, wherein each expression cassette is functionally linked with a promoter and/or enhancer sequence as defined above. Accordingly, this embodiment does not produce just one transcript including both the protein of interest and the inventive fusion protein linked by an IRES sequence. Instead, two transcripts are provided. Such a system may be advantageously used, if the molecular weight of the protein of interest exceeds a critical value. In a preferred embodiment of this alternative, the promoters of the murine CMV immediate early region regulate the expression of genes encoding the protein of interest, and the inventive fusion protein is expressed from an additional expression cassette inserted in the vector backbone. The mCMV(IE1) and mCMV(IE2) promoters may regulate the expression either of two identical copies of the gene encoding the protein of interest, or of two subunits of a multimeric protein of interest such as antibodies or peptide hormones.

A fourth aspect of the invention refers to host cells transfected with an inventive (expression) vector according to the invention. Many cells are suitable for such a transfection in accordance with the present invention, e.g. primary or established cell lines from a wide variety of eukaryotes including plant, yeast, human and animal cells, as well as prokaryotic, viral, or bacterial cells. Preferably, inventive host cells are eukaryotic cells, derived e.g. from eukaryotic microorganisms, such as Saccharomyces cerevisiae (Stinchcomb et al., Nature, 282:39, (1997)). More preferably, cells from multi-cellular organisms are selected as host cells for expression of nucleic acid sequences according to the present invention. Cells from multi-cellular organisms are particularly preferred, if post-translational modifications, e.g.

glycosylation of the encoded proteins, are required (N and/or O coupled). In contrast to prokaryotic cells, higher eukaryotic cells may permit these modifications to occur. The skilled person is aware of a plurality of established cell lines suitable for this purpose, e.g. 293T (embryonic kidney cell line), HeLa (human cervix carcinoma cells) and further cell lines, in particular cell lines established for laboratory use, such as HEK293-, Sf9- or COS-cells or cells of the immune system or adult stem cells, such as stem cells of the hematopoietic system (derived from bone marrow). More preferably, the cell is a mammalian cell. Most preferably, said cell is a cell from Chinese hamster or a human cell. For example, suitable cells include NIH-3T3 cells, COS cells, MRC-5 cells, BHK cells, VERO cells, CHO cells, rCHO-tPA cells, rCHO-Hep B Surface Antigen cells, HEK 293 cells, rHEK 293 cells, rC127-Hep B Surface Antigen cells, CV1 cells, mouse L cells, HT1080 cells, LM cells, Y1 cells, NS0 and SP2/0 mouse hybridoma cells and the like, RPMI-8226 cells, Vero cells, WI-38 cells, MRC-5 cells, Normal Human fibroblast cells, Human stroma cells, Human hepatocyte cells, human osteosarcoma cells, Namalwa cells, human retinoblast cells, PER.C6 cells and other immortalized and/or transformed mammalian cells. Preferably, said vector comprises a sequence encoding a fusion protein comprising SEQ ID NO: 23. Most preferably, said vector comprises a sequence of SEQ ID NO: 22.

A fifth aspect of the present invention refers to a method of screening cells for expression or high expression of a protein of interest, said method comprising the steps of:
(i) transfecting cells with an inventive expression vector;
(ii) selecting cell clones being resistant to an antibiotic as defined above;
(iii) incubating cells selected according to step (ii) with a solution containing the ligand; and
(iv) detecting the fluorescence activity of cell clones selected according to step (ii) due to fluorescence of the ligand.

In step (i) of the inventive cell screening method of screening cells, cells are transfected with an inventive expression vector as defined above. Therefore, the cells to be transfected in step (i) are preferably cells, which upon successful transfection, should express both the inventive fusion protein and the protein of interest (POI). More preferably, cells to be transfected are selected from the cell lines disclosed above. The transfection may be performed by methods known to a skilled person and as described in the prior art, e.g. Sambrook, J. C., Fritsch, E. F., and Maniatis, T. (2001) Molecular Cloning: A Laboratory Manual, Cold Spring Harbor Laboratory Press, Cold Spring Harbor, N.Y. Preferably, said vector comprises a sequence encoding a fusion protein comprising SEQ ID NO: 23. Most preferably, said vector comprises a sequence of SEQ ID NO: 22.

In step (ii) of the inventive cell screening method, cells are selected which are resistant to an antibiotic as defined above, i.e. which were successfully transfected in step (i) and express a peptide conferring a resistance for an antibiotic as defined above (i.e. neomycin, kanamycin, neomycin-kanamycin, hygromycin, gentamycin, chloramphenicol, puromycin, zeocin or bleomycin, respectively). Accordingly, cells are preferably grown, typically for 1 hour up to 3 weeks, in a culture medium under selective conditions, i.e. in the presence of the corresponding antibiotic for exerting selection pressure from the very beginning of cultivation. Alternatively, cells are typically grown for 1 hour up to 3 weeks, in a culture medium under non-selective conditions, and the corresponding antibiotic is preferably added at a predetermined time, e.g. when cells exhibit a specific optical density (OD-value). Suitable cell culturing conditions are preferably those known to a skilled person and as described in the prior art, e.g. Sambrook, J. C., Fritsch, E. F., and Maniatis, T. (2001) Molecular Cloning: A Laboratory Manual, Cold Spring Harbor Laboratory Press, Cold Spring Harbor, N.Y. Most preferably, cells which were successfully transfected express a fusion protein comprising SEQ ID NO: 23 conferring resistance for puromycin.

In subsequent step (iii) cells as selected in step (ii) are typically incubated with a solution containing a membrane-permeable fluorescein derivative 4′,5′-bis(1,3,2-dithioarsolan-2-yl)-fluorescein, or any derivative thereof exhibiting the same binding properties. Thereby the inventive fusion protein is labeled with the ligand (or a derivative thereof) upon binding to its component(s), comprising at least one sequence SEQ ID NO: 1, 2 or 3. Labeling with the ligand may be performed by using the labeling protocol according to Example 2 (see below). Alternatively, the Lumio™In-Cell Labeling Kit from Invitrogen Corporation may be used according to the manufacturers instructions. Similarly, labeling with a derivative of the ligand may be performed according to these protocols.

In final step (iv) fluorescence of the labelled cells is elicited via the acquired fluorescence of the ligand, or a derivative thereof. Fluorescence of the ligand, when bound to any of SEQ ID NO: 1, 2 or 3 of the inventive fusion protein, may be evoked after excitation. The emitted fluorescence spectra can be detected by using any of the above mentioned methods for detecting fluorescence, most preferably by using FACS. The excitation wavelength is typically in a range from 450 to 650 nm and emittance of fluorescent light is typically observed in a range of from 450 to 700 nm.

Any number of cells may be screened by such a method. Preferably, the fluorescence activity of at least 20, 50, 100, 500, 1.000, 5.000, 10.000, 50.000, 100.000, 500.000 or 1.000.000 cells is detected in step (iv). Most preferably, a population of cells sufficient for obtaining at least 1.000 to 10.000.000 independent transfectants being resistant to an antibiotic as defined above is screened. Among these, at least 10 to 1.000.000 candidate clones being resistant to this antibiotic may be sorted by evaluating the fluorescence activity of these cells. Preferably, about 20% of cells that exhibit highest fluorescence activity in step (iv) are selected as cells that exhibit highest expression of said protein of interest. More preferably, the 10% of cells that exhibit highest fluorescence activity in step (iv) comprise the cells that exhibit highest expression of said protein of interest. Even more preferably, the 5% of cells that exhibit highest fluorescence activity in step (iv) comprise the cell that exhibit highest expression of said protein of interest. Preferably, the cells are screened cell by cell using FACS.

In the context of the present invention, “high expression” refers to an expression level in a cell, which is screened, that is higher than in other cells that are screened. “High expression” of a protein is a relative value. For example, final expression levels of recombinant proteins that are commercially produced range from 1 to 2.000 mg/l (cell culture), depending on the protein, annual quantities required and therapeutic dose. During a screening, the expression level of a protein of interest is typically lower than the final expression level.

The cells obtained at the end of the above screening method may be ranked relative to each other regarding the expression level of the protein of interest (POI). Particularly, the cells exhibiting the highest fluorescence activity may be selected at the end of the above method of screening. For example, individual cells exhibiting fluorescence activity corresponding to the top 5-20% of inventive expressors are selected for further analysis of expression of the gene of interest in a subsequent step.

In a preferred embodiment, the above screening method further comprises an optional step (v) comprising selecting about 5% to about 20% of the cells assayed in step (iv), wherein the selected cells are those exhibiting highest fluorescence activity in step (iv). Alternatively, about 5% to about 30%, 40%, 50%, 60%, 70% or 80% of the cells assayed in step (iv) may be selected based on highest activity of the protein of interest. Then, upon selection of the cells exhibiting the highest fluorescence activity, the expression level of the protein of interest in said selected cells may further be determined.

In another preferred embodiment, the above method of screening is performed using multiwell microtiter plates or similar.

A sixth aspect of the present invention refers to a method for obtaining a cell line expressing a protein of interest, said method comprising the step of:
(i) screening cells according to any of the above inventive cell screening methods;
(ii) selecting the cell(s) exhibiting the highest expression of said protein of interest, preferably according to any of the above inventive methods; and
(iii) establishing a cell line from said cell.

As used herein, a “cell line” refers to one specific type of cell that can grow in a laboratory, i.e. cell lines from cells as defined above. A cell line can usually be grown in a permanently established cell culture, and will proliferate indefinitely given appropriate fresh medium and space. Methods of establishing cell lines from isolated cells are well-known by those of skill in the art. Preferably, cell lines are prepared from cells as mentioned above.

A seventh aspect refers to a method of producing a protein of interest, said method comprising the steps of:
(i) culturing a cell line obtained according to an inventive method as described above, under conditions which (selectively) permit expression of said protein of interest; and
(ii) isolating said protein of interest.

Conditions which (selectively) permit expression of the protein of interest can easily be established by one of skill in the art by standard methods. Alternatively, any condition suitable for the protein of interest to be expressed and known to a skilled person may be used. Such methods are disclosed in e.g. Sambrook, J. C., Fritsch, E. F., and Maniatis, T. (2001) Molecular Cloning: A Laboratory Manual, Cold Spring Harbor Laboratory Press, Cold Spring Harbor, N.Y.

In the context of the present invention “isolating” typically comprises purifying the protein of interest. The purification may be carried out by any technique well-known by those of skill in the art, e.g. by conventional biochemical methods, such as chromatography, e.g. affinity chromatography (HPLC, FPLC, . . . ), size exclusion chromatography, etc., as well as by cell sorting assays, antibody detection, etc. or by any method disclosed by Sambrook et al, (2001, supra). In case the protein of interest shall be applied as medicament, it is preferably formulated into a pharmaceutical composition. Preferably, such pharmaceutical compositions comprises the protein of interest as disclosed above. Additionally, such a pharmaceutical composition may comprise a pharmaceutically acceptable carrier, adjuvant, or vehicle according to the invention refers to a non-toxic carrier, adjuvant or vehicle that does not destroy the pharmacological activity of the protein of interest with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Furthermore, an eighth aspect of the present invention refers to a method of producing an inventive fusion protein comprising the steps of:
(i) culturing a cell as defined above (e.g. comprising a nucleic acid encoding the inventive fusion protein) under conditions which (selectively) permit expression of the inventive fusion protein; and
(ii) isolating the inventive fusion protein.

In a preferred embodiment, the nucleic acid encodes a fusion protein comprises the sequence according to SEQ ID NO: 23, or comprises the sequence of SEQ ID NO: 22.

In the context of the present invention “isolating” also comprises purifying the inventive fusion protein, if necessary. The purification may be carried out by any method as disclosed above. Furthermore, such a method may for example be performed e.g. as described in Example 1.

Such a method as disclosed above for producing an inventive fusion protein may be suitable, e.g. for, without being limited, discovering the properties of the inventive fusion protein in vitro, e.g. binding properties of the membrane permeable fluorescein derivative, signal intensity, exhibited upon binding, solubility of the fusion protein under physiologic conditions, etc.

A ninth aspect of the present invention refers to the use of a cell as disclosed above comprising an inventive nucleic acid as disclosed above for producing a protein of interest. Preferably, said inventive nucleic acid is contained in a vector or an expression vector, preferably an (expression) inventive vector as defined above.

A tenth aspect of the invention refers to the use of an inventive fusion protein as defined above, of a nucleic acid according to the present invention or of an inventive (expression) vector for screening cells for expression or for high expression of a protein of interest. Preferably, cells are therefore screened at first in a primary screen for high fluorescence activity. Then, fluorescence activity may be correlated to the expression of a protein of interest by inference. This allows to rapidly eliminate 80 to 95% of the tested cells based on low fluorescence activity, and to retain the remaining 5-20% for analysis of expression of the gene of interest in a step. Most preferably, the inventive fusion protein comprises the sequence according to SEQ ID NO: 23, and/or is encoded by the sequence of SEQ ID NO: 22.

Having now fully described this invention, it will be appreciated by those skilled in the art that the same can be performed within a wide range of equivalent parameters, concentrations and conditions without departing from the spirit and scope of the invention and without undue experimentation.

While this invention has been described in connection with specific embodiments thereof, it will be understood that it is capable of further modifications. This application is intended to cover any variations, uses or adaptations of the invention following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or unpublished patent applications, issued patents or any other references, are entirely incorporated by reference herein, including all data, tables, figures and text presented in the cited references. Additionally, the entire contents of the references cited within the references cited herein are also entirely incorporated by reference. Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### BRIEF DESCRIPTION OF THE FIGURES

FIG. 1: shows the principle of binding of a ligand to SEQ ID NO's: 1, 2 or 3 (1A) in the inventive fusion protein. Just upon binding to any of SEQ ID NO's: 1, 2 or 3, the ligand becomes fluorescent and may be detected using common fluorescence detection methods.

In FIG. 1B, an exemplary bi-cistronic mRNA, encoding the inventive fusion protein and the protein of interest, is disclosed, wherein both coding sequences are separated by an TRES sequence. Linking expression of the gene of interest (p.ex. SEAP) to the fusion protein on a bicistronic mRNA allows correlated expression of both proteins. High expression of the inventive fusion protein is thus correlated with strong fluorescence, and this is indicative for high SEAP production.

FIG. 2: shows transfection of plasmids pmCMV(IE1)-SEAP-IRES-Puro-279 (in more detail disclosed in FIG. 6) and pmCMV(IE1)-SEAP-IRES-PuroLT-280 (in more detail disclosed in FIG. 5) into CHO-S cells (PEI25/in suspension). Selection of stable transfectants using puromycin as a first component leads to recovery of viability up to 100% after 2 or 3 weeks. In a control, wherein cells comprise a plasmid without puromycin resistance, all cells were depleted.

FIG. 3: shows the correlation between SEAP expression levels (upper row) and fluorescence intensity subsequent to labeling with 4′,5′-bis(1,3,2-dithioarsolan-2-yl)fluorescein (lower row) for thirty different clones. The left column (“Low LumioTag”) and middle column (“High LumioTag”) show clones screened using the inventive fusion protein as a bifunctional marker, as described in detail in Example 7. The right column (“HT Screen”) shows clones screened using a classical high-throughput screening approach.

FIG. 4: shows the plasmid map of pSV40-SEAP-IRES-PuroLT-260, having 6303 bp. pSV40-SEAP-IRES-PuroLT-260 comprises a SV40 promoter, a SEAP coding sequence and a sequence, coding for an exemplary inventive fusion protein (herein designated puroLT). Both coding sequences are separated by a poliovirus IRES sequence.

FIG. 5: shows the plasmid map of pmCMV(IE1)-SEAP-IRES-PuroLT-280, having 6638 bp. pmCMV(IE1)-SEAP-IRES-PuroLT-280 differs from pSV40-SEAP-IRES-PuroLT-260 (FIG. 4) in that the mCMV(IE1) promoter was used instead of the SV40 promoter.

FIG. 6: shows the plasmid map of pmCMV(IE1)-SEAP-IRES-Puro-279, having 6613 bp. pmCMV(IE1)-SEAP-IRES-Puro-279 differs from pmCMV(IE1)-SEAP-IRES-PuroLT-280 in that the sequence encoding puromycin N-acetyl transferase was used instead of the sequence for the inventive fusion protein. pmCMV(IE1)-SEAP-IRES-Puro-279 preferably serves as a negative control in the experiments.

FIG. 7: shows the plasmid map of pmCMV(IE1)-PuroR-LT-273, having 4435 bp. pmCMV(IE1)-PuroR-LT-273 differs from pmCMV(IE1)-SEAP-IRES-Puro-279 in that the coding sequence for SEAP and the IRES sequence are missing. pmCMV(IE1)-SEAP-IRES-Puro-279 also serves as a control in the experiments.

FIG. 8: depicts labeling with the ligand (here 4′,5′-bis(1,3,2-dithioarsolan-2-yl)fluorescein) and transient transfections in CHO cells. As may be seen from the experiments, temperature is shifted prior to staining. Furthermore, the inventive fusion protein will be detected if the expression level is high enough. Following results were obtained for the inventive constructs:

| | at 37° C. | at 29° C. |
|---|---|---|
| pmCMV(IE1)-SEAP-IRES-PuroLT-279 | +++ | +++ |
| pmCMV(IE1)-SEAP-IRES-PuroLT-280 | − | ++ |

The expression level of pmCMV(IE1)-SEAP-IRES-PuroLT-279 thus showed no temperature shift. However, a temperature shift was observed for expression of pmCMV(IE1)-SEAP-IRES-PuroLT-280, comprising the inventive fusion protein with SEAP and IRES sequences. Higher PuroLT levels at 29° C. in this respect could result from increased transcription or IRES activity, mRNA or protein stability. As a conclusion, the induction times from o/n to 24 hr were sufficient.

FIG. 9: depicts the mean fluorescence intensity level (MFI) after labeling with 4′,5′-bis(1,3,2-dithioarsolan-2-yl)fluorescein, measured by FACS, of CHO cells transfected with the pmCMV(IE1)-SEAP-IRES-PuroLT-280 plasmid. Successive sorting of the cells using a Becton-Dickinson FACS, based on high fluorescence, allowed obtaining populations of cells exhibiting increased MFI.

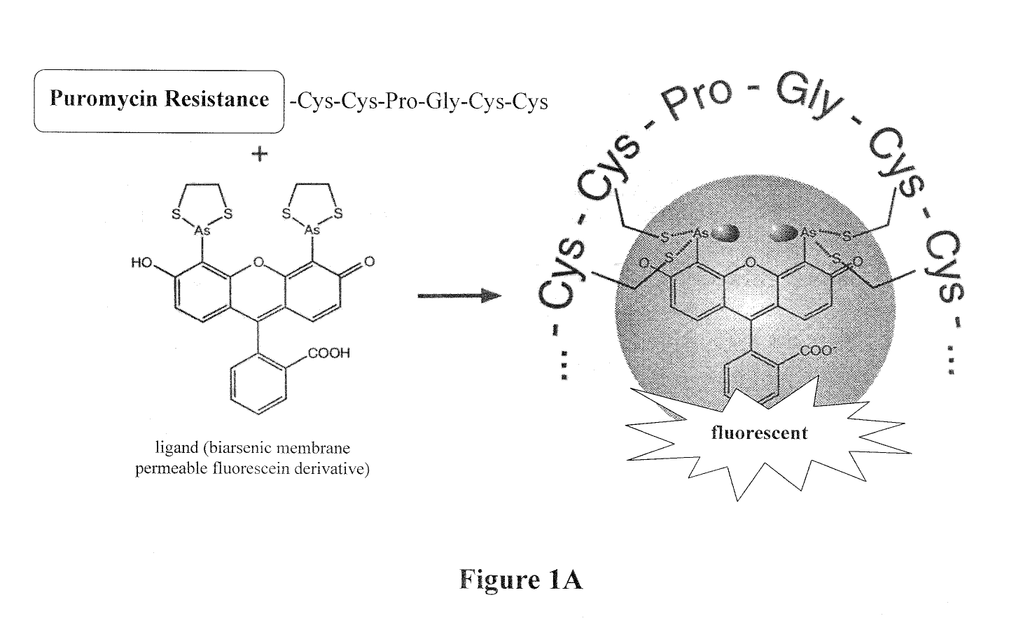

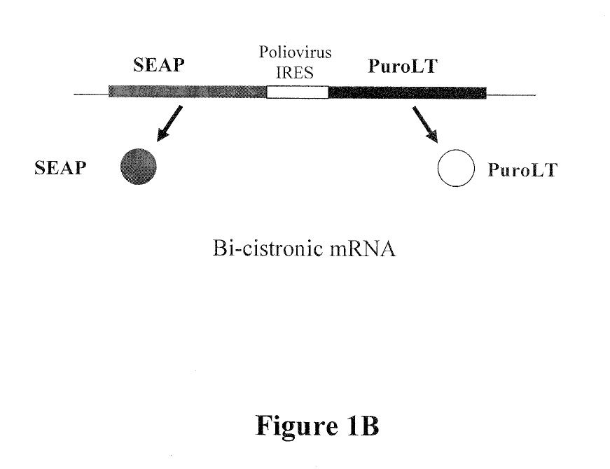

### BRIEF DESCRIPTION OF THE SEQUENCES OF THE SEQUENCE LISTING

SEQ ID NO: 1 corresponds to the generic binding sequence of the ligand of SEQ ID NOs: 1, 2 or 3.

SEQ ID NO: 2 corresponds to a more specific binding sequence of the ligand of SEQ ID NOs: 1, 2 or 3, wherein amino acids at positions 3 and 4 in SEQ ID NO: 2 are defined as Proline and Glycine, respectively.

SEQ ID NO: 3 corresponds to a more specific binding sequence of the ligand of SEQ ID NOs: 1, 2 or 3, which is extended N- and C-terminally with respect to SEQ ID NO: 2.

SEQ ID NOs: 4,5 correspond to the resistance gene for the antibiotic puromycin and the encoded puromycin N-acetyltransferase of Streptomyces alboniger pac.

SEQ ID NOs: 6,7 correspond to the resistance gene for the antibiotic neomycin and the encoded neomycin phosphotransferase type II.

SEQ ID NOs: 8,9 correspond to the resistance gene for the antibiotic kanamycin and the encoded kanamycin phosphotransferase type II.

SEQ ID NOs: 10,11 correspond to the resistance gene for the antibiotic neomycin-kanamycin and the encoded neomycin-kanamycin phosphotransferase type II.

SEQ ID NOs: 12,13 correspond to the resistance gene for the antibiotic hygromycin and the hygromycin phospho transferase.

SEQ ID NOs: 14,15 correspond to the resistance gene for the antibiotic gentamycin and the encoded gentamycin acetyl transferase.

SEQ ID NOs: 16,17 correspond to the resistance gene for the antibiotic chloramphenicol and the encoded chloramphenicol acetyltransferase.

SEQ ID NOs: 18,19 correspond to the resistance gene for the antibiotic zeocin and the encoded zeocin resistance protein.

SEQ ID NOs: 20,21 correspond to the resistance gene for the antibiotic bleomycin and the encoded bleomycin resistance protein.

SEQ ID NOs: 22,23 correspond to the nucleic acid sequence encoding an exemplary inventive chimeric selection marker and the inventive chimeric selection marker.

SEQ ID NOs: 24,25 correspond to primers oSerono1206 and oSerono1239, used for constructing an exemplary inventive fusion protein.

### EXAMPLES

### 1. Example 1

### Construction of an Exemplary Inventive Fusion Protein by PCR

A gene encoding the fusion protein, comprising puromycin N-acetyl transferase (pac) and SEQ ID NO: 3, and a protein of interest (here SEAP, secreted alkaline phosphatase) was constructed by fusing the open reading frame for puromycin N-acetyl transferase (pac) fused to SEQ ID NO: 3, by PCR cloning into an expression vector comprising a first open reading frame encoding SEAP, followed by a poliovirus IRES. The poliovirus IRES sequence allows separating two open reading frames, which are expressed from the same promoter but as two separate proteins.

1.1. Cloning of Nucleic Acid for pSV40-SEAP-IRES-PuroLT-260

Therefore, a gene encoding a fusion of a peptide (-GCCPGCCGGG, SEQ ID NO: 3) to the C-terminus of the puromycin resistance gene was created by the polymerase chain reaction (PCR) using oligos oSerono1206 (5′-GTGGCTGCTTATGGTGACAATC-3′, SEQ ID NO: 24) and oSerono1239 (5′-CGCGCTAGCTCATTACTAGCCGCCACCGCAACAGCCAGGACAACAGCCGGCA CCGGGCTTGCGGGTC-3′, SEQ ID NO: 25). The resulting gene (designated PuroLT) was cloned into the pSV40-SEAP-IRES-puro-227 vector, which confers resistance to puromycin and comprises the SEAP open reading frame under the control of the SV40 promoter. The resulting plasmid was referred to as pSV40-SEAP-IRES-PuroLT-260. The inserted fragment was verified by sequencing.

The SV40 promoters of pSV40-SEAP-IRES-puro-227 and pSV40-SEAP-IRES-PuroLT-260 were replaced with the murine CMV IE1 promoter (mCMV(IE1), described e.g. in WO 87/03905) to generate pmCMV(IE1)-SEAP-IRES-Puro-279 and pmCMV(IE1)-SEAP-IRES-PuroLT-280, respectively.

The PCR conditions were as follows:
Amplification of pac: 25 pmol of primers of SEQ ID Nos. 24 and 25 were mixed with about 20 ng of the XbaI/MfeI fragment from a vector comprising the pac open reading frame, 200 M of each dNTPs, 1× KOD, 2 units of KOD DNA polymerase (KOD Hot Start DNA polymerase, catalogue No. 71086-3, Novagen). The final volume was of 100 μl.
Cycling:
   First step: 3 minutes at 94° C.
   12 cycles: (i) denaturation of 15 seconds at 94° C.; (ii) hybridization of 15 seconds at 55° C.; and (iii) polymerization of 1 minute at 72° C.;
   Final step: 7 minutes at 72° C.

The obtained PCR product for puroLT was firstly analyzed by PAGE analysis. Each PCR reaction was purified using the QIAquick PCR purification kit (Catalog No. 28106, Qiagen) following manufacturer's protocol.

For cloning into the pSV40-SEAP-IRES-puro-227 vector, the PCR fragment was purified using the MinElute Gel Extraction kit (Catalog No. 28606, Qiagen) following manufacturer's protocol.

1.2. Cloning of Nucleic Acid for pmCMV(IE1)-SEAP-IRES-PuroLT-280

Subsequent to verifying the pSV40-SEAP-IRES-puroLT-260 vector sequence (see 1.1) the SV40 promoter sequence was replaced by the murine CMV promoter to generate pmCMV(IE1)-SEAP-1RES-PuroLT-280.

1.3 Cloning of Nucleic Acid for pmCMV(IE1)-SEAP-IRES-Puro-279

Cloning of nucleic acid for pmCMV(IE1)-SEAP-IRES-PuroLT-279 was carried out similar as disclosed above for pmCMV(IE1)-SEAP-IRES-PuroLT-280, wherein the nucleic acid encoding puromycin N-acetyl transferase (puro) was cloned into the vector instead of the nucleic acid encoding the inventive fusion protein.

### 2. Example 2

### Labeling Protocol with the Ligand

The inventive fusion protein was labeled with the ligand using following protocol.
Cells were firstly washed 1× with HBSS (Hanks balanced salt solution, Gibco cat #14025-050). If cells were used grown in ProCHO5—Pluronic acid at 0.05% final concentration was included.
Cells were incubated cells in 1× Labeling Solution for 30 minutes at room temperature in the dark. The 1× Labeling Solution comprises HBSS (Gibco cat #14025-050). 1 μM 4′,5′-bis(1,3,2-dithioarsolan-2-yl)fluorescein and 50 μM EDT (1,2-Ethandithiol Sigma cat #39,802-0). If cells were used, which were grown in ProCHO5 Pluronic acid at 0.1% final concentration was added.
The Labeling Solution was removed (and discarded appropriately). The cells were then washed once in HBSS+50 μM EDT. If cells were used, which were grown in ProCHO5, Pluronic acid at 0.1% final concentration was used.
Cells were then added or resuspended in HBSS+20 μM Disperse Blue 3 (supplied with LumioGreen Kit Invitrogen cat #45-7510). If cells were used, which were grown in ProCHO5, Pluronic acid at 0.1% final concentration was included.

In order to detect fluorescence of a ligand to SEQ ID NO: 1, 2 or 3 in cells expressing the inventive fusion protein, the cells were pre-incubated o/n to 24 hr at 29° C. prior to labeling with 4′,5′-bis(1,3,2-dithioarsolan-2-yl)fluorescein.

After labeling with 4′,5′-bis(1,3,2-dithioarsolan-2-yl)fluorescein, the cells were observed under a fluorescence microscope (Olympus CKX41 microscope equipped with a DP50 digital camera) using a standard ETC filter set.

### 3. Example 3

### Transfection of Vectors

### 3.1. Transfection of Vectors Encoding the Inventive Fusion Protein

One day before transfection, cells grown in ProCHO5 medium were passaged at 0.75×10⁶ cells/ml. Just before transfection, 8-10×10⁶ cells were centrifuged, washed with RPMI 1640+ Glutamax, resuspended in 15 ml of the same medium and distributed in 6w plates (2.5 ml/well) or 24w plates (0.5 ml/well).

Linear PEI25 (MW25000, Polysciences, Cat. #23966) was used as transfecting agent at 3-3.5 μl of 1 mg/ml PEI25 solution per μg of DNA. The PEI25 1 mg/ml solution was filter sterilised, aliquoted in 1 ml fractions and kept at −70° C.

Plasmid DNA in 150 mM NaCl was mixed with PEI25, incubated 10 min at RT and added to the cells. After 2 hours at 37° C., transfection medium was removed and replaced by 3 ml of ProCHO5 supplemented with 4 mM glutamine and 1× HT. Plates were incubated o/n at 37° C. with shaking at 60 rpm. Cells were pooled from all the wells and plated at 0.5×10⁶ cells/ml of ProCHO5 (supplemented with 4 mM glutamine and 1× HT) in P150 Petri dishes. 48 hours post-transfection cells were counted, spent medium was removed by centrifugation, and cells were then diluted to 1.0×10⁶ viable cells/ml in selection medium (ProCHO5 supplemented with 4.5 mM L-glutamine, 1× HT and 10 μg/ml of puromycin). The medium was changed every other day. Cell densities were monitored over time and, when the number of viable cells dropped below 0.1×10⁶ cells/ml, the cells were concentrated in a smaller volume. Otherwise, when the number of viable cells increased, cells were diluted to 0.4-0.5×10⁶ cells/ml. This procedure was repeated until the viability of the pool reached 90%.

The pool, selected and transfected with plasmid pmCMV(IE1)-SEAP-IRES-PuroLT-280, was seeded at 1 cell/well in 4 384 well plates in ProCHO5 medium supplemented with 4 mM glutamine, 1× HT and 10 μg/ml of puromycin. 176 clones were recovered in 96 well plates.

### 3.2. Selection for Resistance to Puromycin

Cells were transferred to a 15 ml Falcon tube, centrifuged, and the cell pellet was resuspended in 2 ml medium containing 5% Fetal Bovine Serum (FBS) in a 6 well plate. Selection was applied 48 hours post transfection, by exchanging the medium for ProCHO5/HT/Glutamine/5% FBS containing 10 μg/ml of puromycin (Sigma, P-8833). Every two days, a medium exchange was performed by discarding the old medium, washing with 1× PBS, and adding fresh selective medium. After 2 weeks of selection, the cells were trypsinized, counted, and a series of dilutions corresponding to 1000, 500, 100, 50, 20, 10 cells/well of a 6-w format was performed. Ten days later, the colonies growing in all dilutions were counted, and all of them were picked to allow growth in suspension in the absence of serum for clone analysis.

From the results it was concluded that the puromycin resistance conferred by the fusion protein is comparable to the puromycin resistance conferred by the wild-type puromycin resistance gene. In conclusion, the inventive fusion shows the combined activity and function of both SEAP and pac containing fusion protein.

### 4. Example 4

### Measurement of SEAP Expression Levels and Cell Titer Assay

### 4.1. SEAP Assay (Pierce Phosphatase Substrate Kit cat #37620)

100 μl of 1× Phosphatase Substrate Solution were added to 10 of diluted cell-culture medium containing SEAP (diluted 1/10 in HBSS Gibco cat #14025-050).
1× Phosphatase Substrate Solution: (Pierce Phosphatase Substrate Kit cat #37620)
   4 ml H₂O
   1 ml 5× Concentrate Diethanolamine Substrate Buffer
   1 PNPP Substrate Tablet

The solution was then incubated for 10-20 min at 37° C. The OD was read on a Spectrophotometer microplate reader at 405 nm.

### 4.2. Cell Titer Assay (Promega CellTiter96 Aqueous One Cell Proliferation Assay cat #G3580)

20 μl of CellTiter 96Aqueous One Solution Reagent (Promega cat #G3580) were added to 50 μl of cell suspension in 96-well plate 50 μl of RPMI1640 (Gibco cat #61870-010). The solution was mixed, incubated for 20-30 min at 37° C. and the OD was read on a Spectrophotometer microplate reader at 490 nm.

### 5. Example 5

### SEAP IIT Screening

Cells to be analyzed were transferred to a 96 well plate (5000-20000 cells per well) in ProCH05/4.5 mM L-Glutamine/10% Fetal Calf Serum and were incubated overnight at 37° C. to allow them to attach to the bottom of the well.

On the next day the cells were washed 2× in ProCHO5/4.5 mM L-Glutamine and pulsed in 150 μl of the same medium for 24 h at 37° C. after which the supernatant was harvested.

### 5.1. Measuring SEAP Expression Levels

10 μl of diluted supernatant ( 1/10 in HBSS) were added to 100 μl of phosphatase substrate solution (Pierce cat #37620) in a 96 well plate. The plate was incubated at 37° C. for 10-15 minutes and OD was read at 490 nm.

### 5.2. Cell Titer Assay

After the pulse, the medium was replaced by a mix of 100 μl of RPMI1640 medium (Gibco cat #61870-010) plus 20 μl of CellTiter 96 Aqueous One Solution (Promega cat #G3580) and incubated at 37° C. for 30 minutes. The OD was read at 490 nm.

The clones were ranked according the ratio of SEAP OD at 490 nm/CellTiter OD at 490 nm.

### 6. Example 6

### Dual Function of the Inventive PuroLT Fusion Protein

CHO cells were transfected either with pmCMV(IE1)-SEAP-IRES-puro-279 or with pmCMV(IE1)-SEAP-IRES-PuroLT-280 as described in Example 3.1. Non-transfected cells were used as a control.

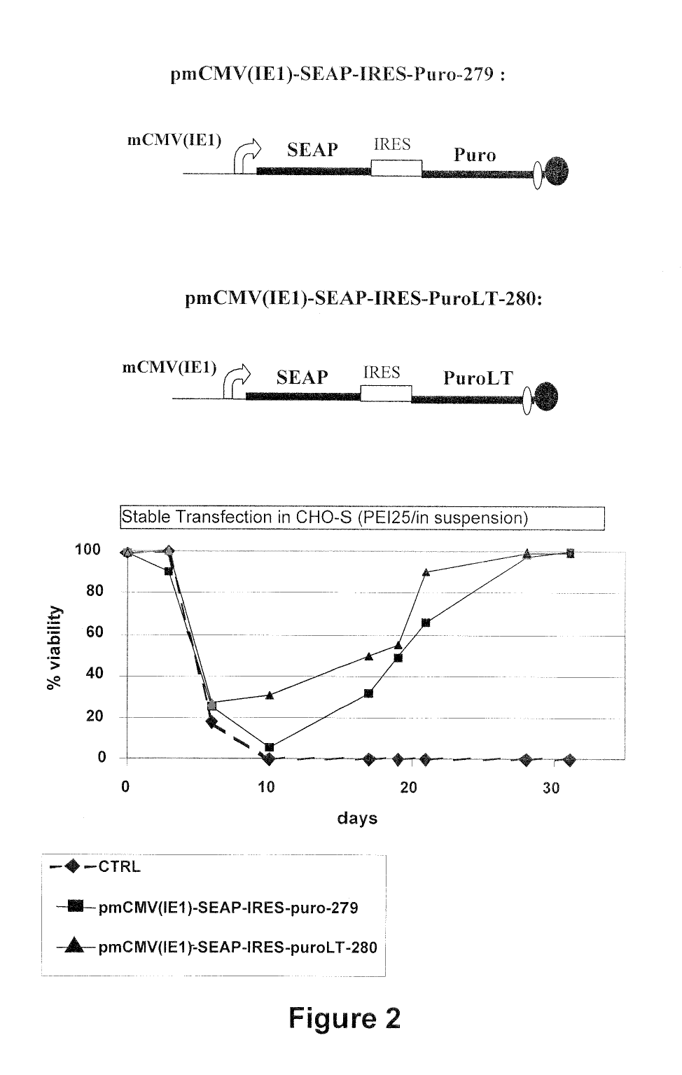

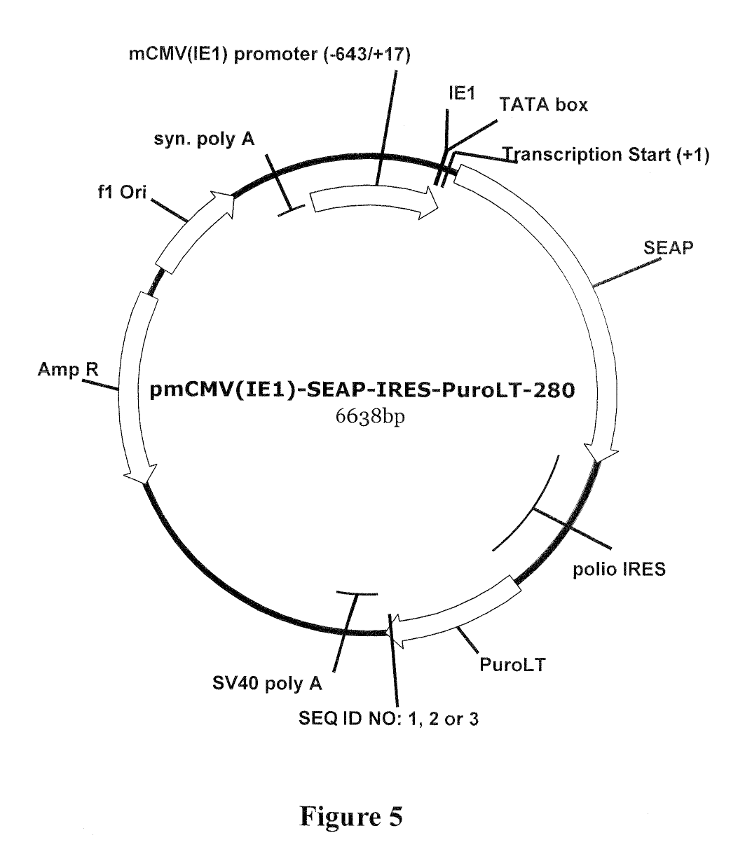

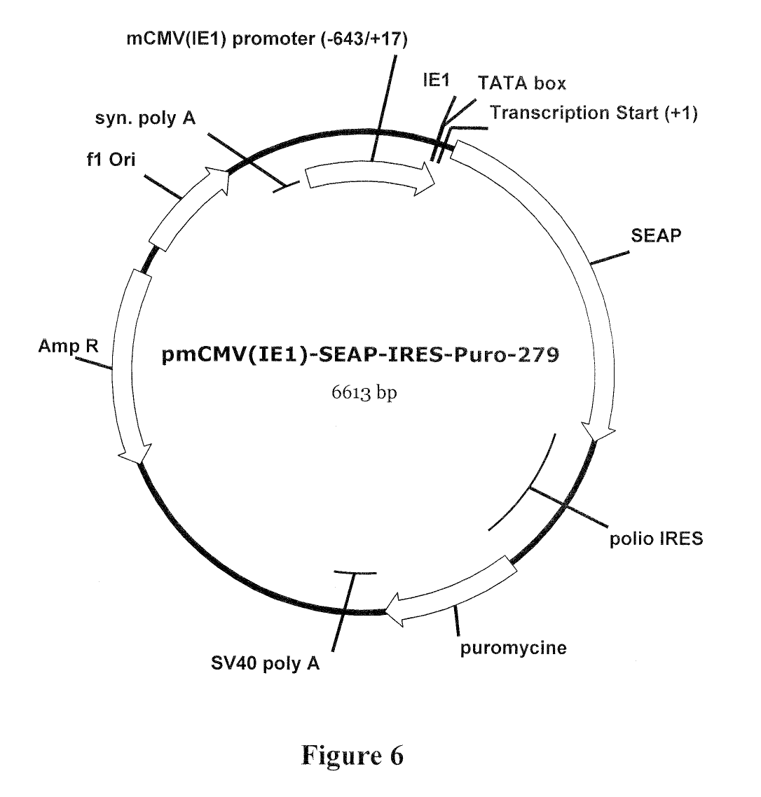

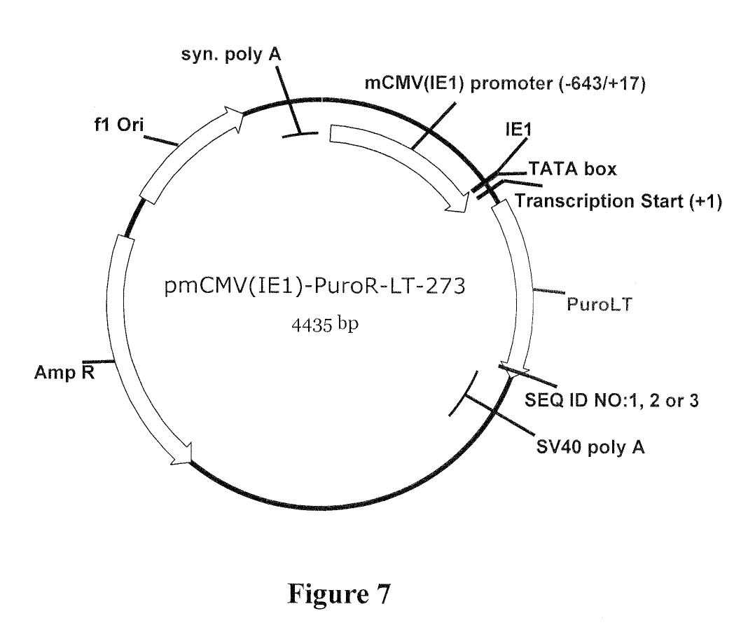

Upon selection with puromycin, pools of viable cells were obtained from the cells transfected either with pmCMV(IE1)-SEAP-IRES-puro-279 or with pmCMV(IE1)-SEAP-IRES-PuroLT-280 (see FIGS. 2, 5 and 6). To the contrary, no viable cells were obtained from the non-transfected cells. This demonstrated that puroLT conferred resistance to puromycin.

The cells transfected either with pmCMV(IE1)-SEAP-IRES-puro-279 or with pmCMV(IE1)-SEAP-IRES-PuroLT-280 were labeled with 4′,5′-bis(1,3,2-dithioarsolan-2-yl)fluorescein as described in Example 2. The cells were either pre-incubated at 37° C. or at 29° C. before labeling.

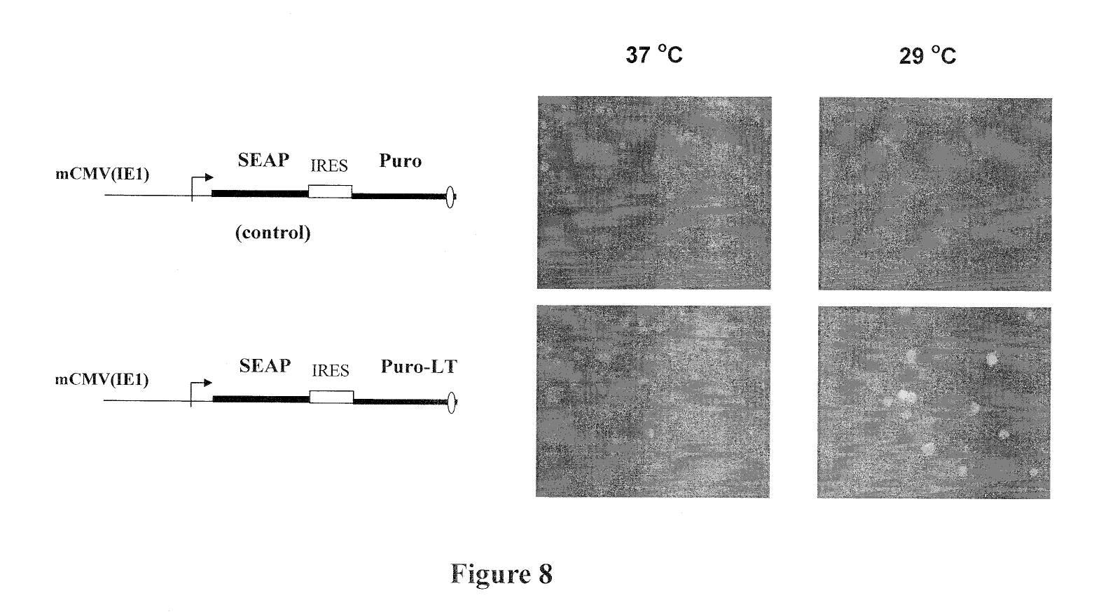

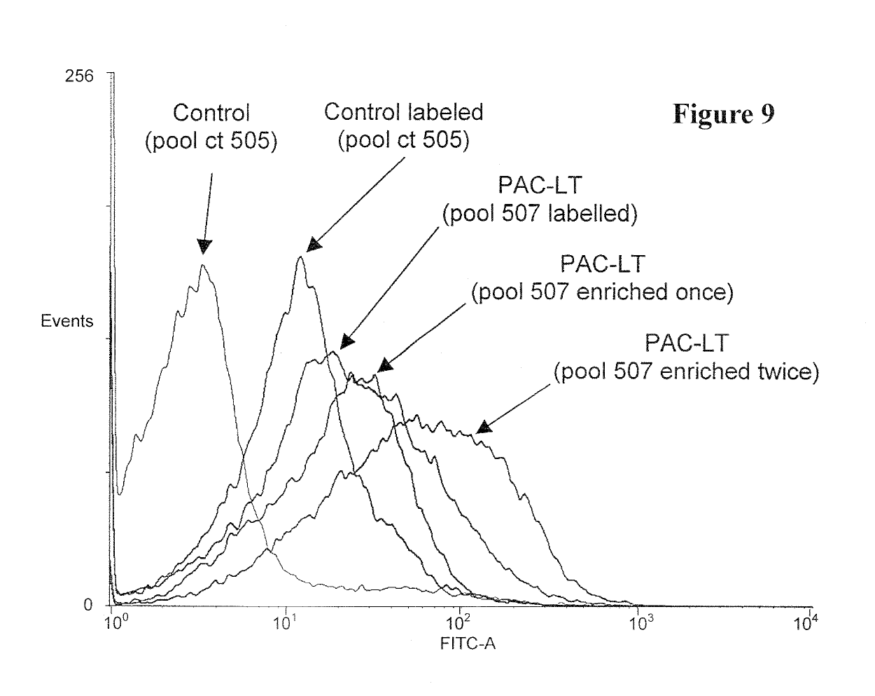

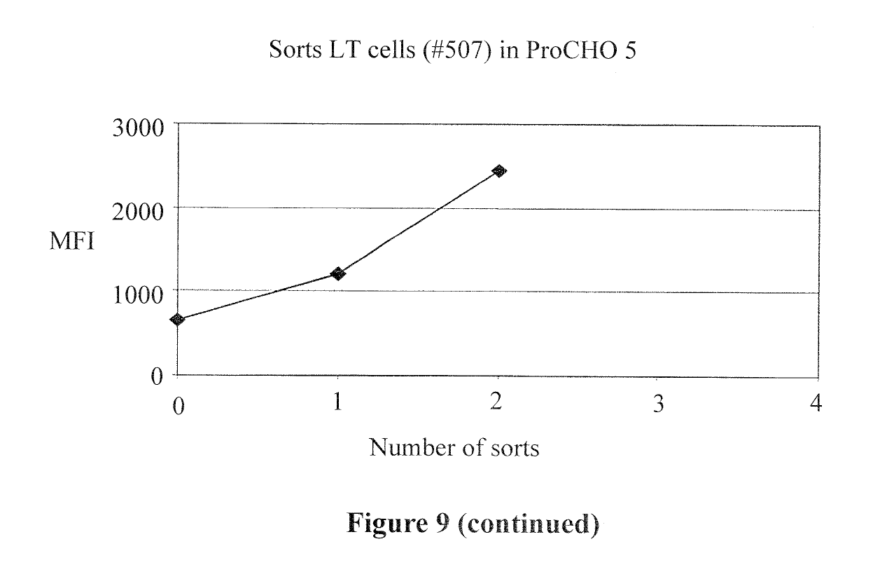

The results are shown in FIG. 8. No fluorescence was detected for cells transfected with pmCMV(IE1)-SEAP-IRES-puro-279, neither when the cells were pre-incubated at 29° C., nor when the cells were pre-incubated at 37° C. To the contrary, fluorescence was detected for cells transfected with pmCMV(IE1)-SEAP-IRES-puro-279 when the cells were preincubated at 29° C. This demonstrated that puroLT becomes fluorescent upon binding to 4′,5′-bis(1,3,2-dithioarsolan-2-yl)fluorescein.

In conclusion, it was demonstrated that puroLT combines the functional properties of pac and of fluorescence upon binding with 4′,5′-bis(1,3,2-dithioarsolan-2-yl)fluorescein. Accordingly, the inventive “puroLT” marker can be used both as a selectable marker in transfections due to its pac activity and as an easily detectable marker due to its fluorescence activity.

### 7. Example 7

### Use of PuroLT as a Bifunctional Marker for Screening Cells for High Expression of a Protein of Interest

The dual function of the created fusion protein suggests that it should also have a dual impact. First, the inventive fusion protein should allow the isolation of stably transfected clones by their resistance to puromycin, and secondly, expression levels of said fusion should reflect expression levels of a physically connected gene of interest by measurement of fluorescence activity. In order to test this hypothesis, a series of clones from pools of cells stably transfected with inventive vectors were generated. Fluorescence activity and expression levels of the encoded proteins were measured.

CHO Cells were transfected with pmCMV(IE1)-SEAP-IRES-PuroLT-280 as described in Example 3.1. 176 clones were obtained The clones were either screened using a classical high-throughput screening as described in Example 5, or labeled with 4′,5′-bis(1,3,2-dithioarsolan-2-yl)fluorescein and visually selected for fluorescence intensity under a fluorescence microscope.

Eight clones expressing high levels of SEAP were selected using the classical high-throughput screening (referred to as “HT Screen”). Twelve highly fluorescent clones and ten moderately fluorescent clones were visually selected based on fluorescence intensity upon labeling with 4′,5′-bis(1,3,2-dithioarsolan-2-yl)fluorescein (respectively referred to as “High LumioTag” and “Low LumioTag”).

The High LumioTag and Low LumioTag clones were further tested for SEAP expression as described in Example 4.

The HT Screen clones were further labeled with 4′,5′-bis(1,3,2-dithioarsolan-2-yl)fluorescein and examined under a fluorescence microscope.

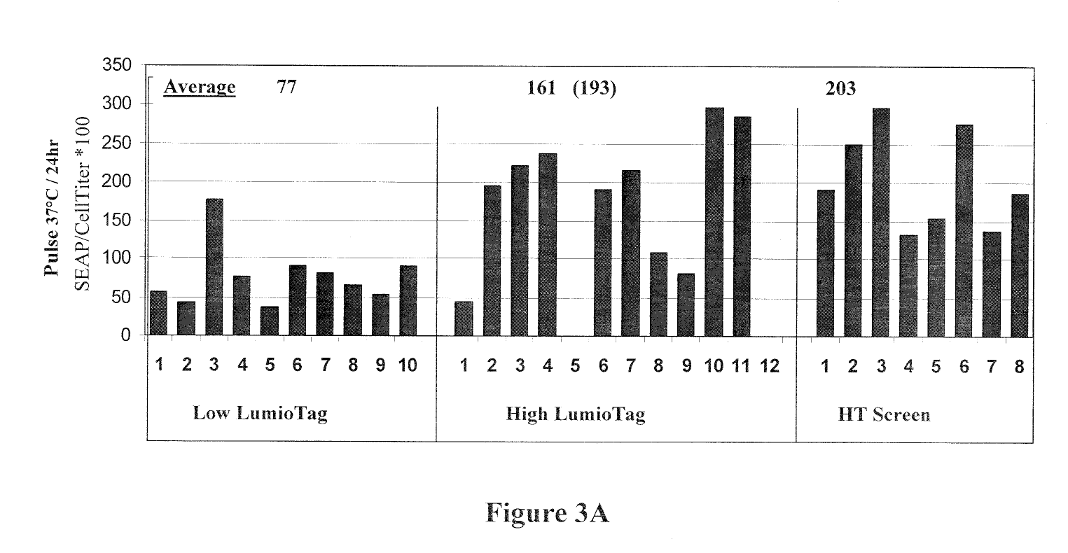

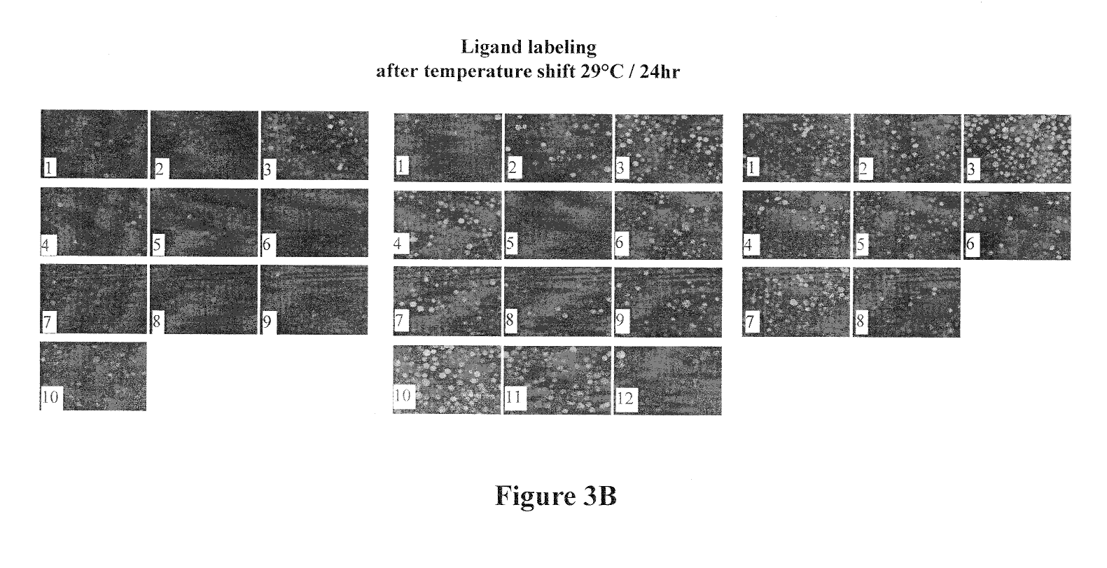

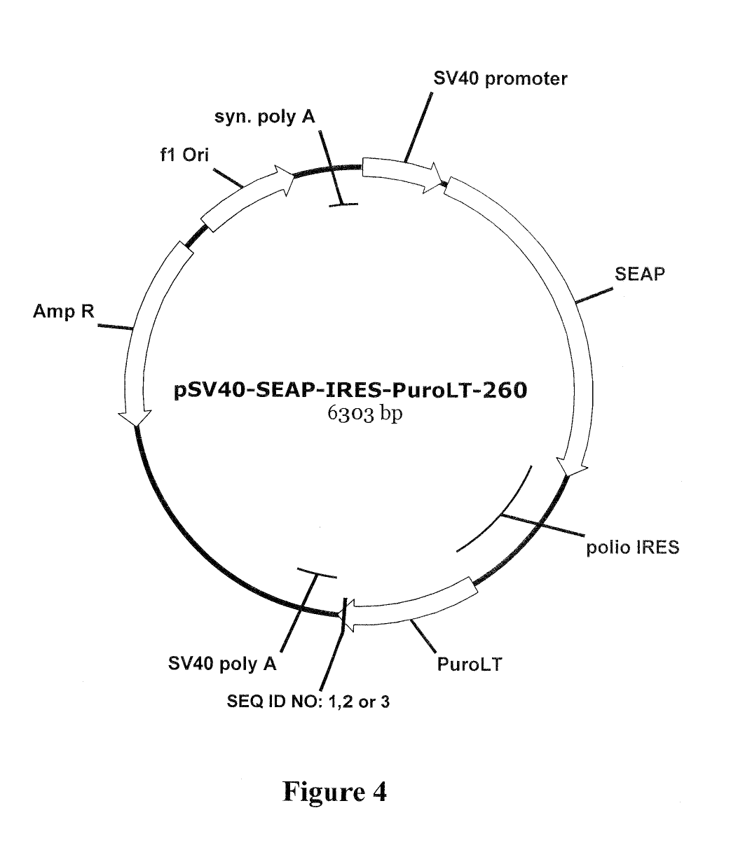

SEAP expression levels and fluorescence intensity obtained for clones selected either using a classical high-throughput screening or for fluorescence intensity upon labeling with 4′,5′-bis(1,3,2-dithioarsolan-2-yl)fluorescein were compared. The results are shown in FIG. 3.

This experiment demonstrates that high SEAP expression level was always correlated with high fluorescence. For example, the High LumioTag clone No. 10 and the HT Screen clone No. 3 exhibit both higher fluorescence and higher SEAP expression level than the other clones.

This experiment further demonstrates that screening using the inventive fusion protein upon labeling with 4′,5′-bis(1,3,2-dithioarsolan-2-yl)fluorescein allows to isolate clones that are as good SEAP expressors than those isolated using a standard high-throughput screening for SEAP expression levels

### 8. Example 8

### Screening for High Fluorescence Expression Using a FACS

In example 7, the screening for highly fluorescent clones was performed manually using a fluorescence microscope. The present experiment shows that the screening for highly fluorescence clones can be made automatically using a Fluorescense Activited Cell Sorter (FACS).

CHO cells were transfected with the pmCMV (IE1)-SEAP-IRES-PuroLT-280 as described in example 3.1, and a pool of cells referred to as “nb 507” was obtained. A control pool (referred to as “nb 505”) was generated using a control in which the puromycin gene was not fused to the Lumio-Tag (plasmid pmCMV(IE1)-SEAP-IRES-puro-279).

To select a highly fluorescent subpopulation of cells, the two pools were labeled as described in example 2, and were subjected to first analysis and then eventually to successive enrichment for high fluorescence level using a Becton-Dickinson FACS (FACSAria™ cell sorting system). The person skilled in the art knows that highly fluorescent clones could also be directly selected using a FACS equipped with an automated single cell deposition unit (ACDU).

As shown in FIG. 8, a population of cells showing higher mean fluorescence intensity level (MFI) after labeling with 4′,5′-bis(1,3,2-dithioarsolan-2-yl)fluorescein can be observed by flow cytometry. This population of cells showing higher MFI can further be enriched by sorting. The mean fluorescence of the enriched population increases after successive sorting.

This experiment demonstrates that the enrichment procedure based on high fluorescence and automatic sorting using a FACS correlates with higher average pool expression levels of the chimeric marker. It is expected that in this experiment, high expression levels of the chimeric marker is reflected by high expression levels of the POI, as was the case in the experiment of example 7.

### ADVANTAGES

The present invention refers to a novel chimeric selection marker corresponding to a fusion protein comprising a peptide conferring resistance to an antibiotic, or a fragment, allelic variant, splice variant or mutein thereof, fused to at least one sequence comprising SEQ ID NO: 1, 2 or 3, wherein said fusion protein exhibits: (i) a resistance to said antibiotic; and (i) a fluorescence activity upon binding to a ligand of SEQ ID NO: 1, 2 or 3.

It has been demonstrated that the inventive fusion combines the functional properties of fluorescence measurement and of antibiotic selection (e.g. pac, see Example 2). Accordingly, the inventive marker can be used both as a selectable marker in stable transfections due to its antibiotic resistance and as an easily detectable marker due to its fluorescence activity.

Using the inventive fusion protein in HTS allows furthermore keeping at least the same chance for selecting high expressing clones as when screening using a low-throughput method allowing to directly detect expression of the POI such as, e.g., labeled antibodies. Thus the inventive fusion protein in HTS allows to reduce time and resources. In a classical HTS clone generation approach, the best clones are typically chosen on the basis of high titers for secreted proteins upon screening of more than 2,000 clones. Using the inventive fusion protein particularly leads to a reduction in sample size. This reduction may relate to the ease of use of the inventive approach and the associated reduction of sampling errors and assay variance related to ELISA high throughput screens. In addition, by selecting the 5 to 10 best clones per plate, the best clone per plate is expected to be selected. Thus, using the inventive fusion protein for screening 1,000 clones will reduce the number of clones to be analyzed to 50 to 100, and thus allow the avoidance of a second HTS.

In addition, it is important to note that the POI, expressed in correlation with the inventive fusion protein, is not limited in its size, since fusion of a peptide, conferring resistance to an antibiotic, or a fragment, allelic variant, splice variant or mutein thereof, to a sequence comprising SEQ ID NO: 1, 2 or 3, leads to a small and thus efficient expression cassette. Furthermore, the two individual enzymes with so different activities and origins surprisingly retain their function in the inventive fusion protein as it is described here. The retained dual function clearly leads to a dual impact as the inventive fusion protein can truly be used to provide selectivity in stable transfection and acts as a chimeric selection marker for screening candidate clones for high expression of a gene of interest.

Summarizing the above, the usefulness of the inventive additional selection marker for the isolation of high-expressing clones for a protein of interest (POI), e.g. a therapeutic protein, has been demonstrated. It allows reducing time, cost and resources since (i) standardized product-independent and simple analysis is performed; and (ii) measuring fluorescence activity is an inexpensive assay. The present invention thus provides a powerful marker, which can both be used to provide selectivity in stable transfection and act as a detectable marker for screening candidate clones for high expression of a gene of interest.

### REFERENCES

1. Altschul et al., (1990), J. Mol. Biol. 215, 403-410;
2. Ausubel et al., (1987, 1992), Current Protocols in Molecular Biology, supra, Interscience, N.Y., 6.3 and 6.4;
3. Blackwood and Kadonaga (1998), Science 281, 61-63;
4. Borth et al. (2000); Biotechnol. Bioeng. 71, 266-273;
5. Chesnut et al. (1996); J. Immunol. Methods 193, 17-27;
6. Devereux et al., (1984), Nucleic Acids Res. 12, 387-395;
7. Grantham, R. (1974), Science 185, 862-864;
8. Griffin et al. (1998) Science, July 10; 281 (5374): 269-72);
9. Kaufman et al. (1985), Mol. Cell Biol. 5, 1750-1759;
10. Li, Harju and Peterson, (1999), Trends Genet. 15, 403-408;
11. Messerle, Keil, and Koszinowski, (1991), J. Virol. 65, 1638-1643;
12. Pearson (1990), Methods Enzymol. 183, 63-98;
13. Pearson and Lipman (1988), Proc. Natl. Acad. Sci. U.S.A 85, 2444-2448;
14. PCT/EP2004/050280;
15. Sambrook, J. C., Fritsch, E. F., and Maniatis, T. (2001) Molecular Cloning: A

Laboratory Manual, Cold Spring Harbor Laboratory Press, Cold Spring Harbor, N.Y.);
16. Seliger and McElroy, (1960), Arch. Biochem. Biophys. 88, 136-141);
17. Smith and Waterman, (1981), J. Mol. Biol. 147, 195-197
18. Stinchcomb et al., (1997) Nature, 282:39;
19. U.S. Pat. No. 4,959,314 (Mark et al);
20. U.S. Pat. No. 4,588,585 (Mark et al);
21. U.S. Pat. No. 4,737,462 (Mark et al);
22. U.S. Pat. No. 5,116,943 (Koths et al.);
23. U.S. Pat. No. 4,965,195 (Namen et al.);
24. U.S. Pat. No. 4,879,111 (Chong et al.);
25. U.S. Pat. No. 5,017,691 (Lee et al.);
26. U.S. Pat. No. 4,904,584 (Shaw et al.);
27. US patent publication 2004/0115704;
28. WO 87/03905;
29. WO 01/53325;
30. WO 2006/058900; and
31. Wood, de Wet, Dewji, and DeLuca, (1984), Biochem Biophys. Res. Commun. 124, 592-596.

We claim:

1. A vector comprising a nucleic acid sequence encoding a fusion protein, said fusion protein comprising a peptide sequence conferring resistance to an antibiotic fused to at least one sequence comprising SEQ ID NO: 1, 2 or 3, wherein said fusion protein exhibits:
(i) a resistance to said antibiotic; and
(ii) a fluorescence activity upon binding of a ligand to said sequence comprising SEQ ID NO: 1, 2 or 3.

2. The vector according to claim 1, wherein the peptide sequence confers resistance to an antibiotic selected from neomycin, kanamycin, neomycin-kanamycin, hygromycin, gentamycin, chloramphenicol, puromycin, zeocin or bleomycin.

3. The vector according to claim 1, wherein said peptide conferring resistance to an antibiotic is selected from a sequence at least 85%, 90%, 95%, 96%, 97%, 98% or 99% identical to any of SEQ ID NOs: 5, 7, 9, 11, 13, 15, 17, 19 or 21.

4. The vector according to claim 1, wherein said peptide conferring resistance to an antibiotic is encoded by a sequence at least 85%, 90%, 95%, 96%, 97%, 98% or 99% identical to any of SEQ ID NOs: 4, 6, 8, 10, 12, 14, 16, 18 or 20.

5. The vector according to claim 1, wherein said peptide conferring resistance to an antibiotic is a puromycin N-acetyl transferase (pac) from Streptomyces alboniger.

6. The vector according to claim 5, wherein said puromycin N-acctyl transferase (pac) comprises amino acids 2 to 199 of SEQ ID NO: 5.

7. The vector according to claim 1, wherein the 3′ terminus of said SEQ ID NO: 1, 2 or 3 is fused to the 5′ terminus of said peptide sequence conferring resistance to an antibiotic.

8. The vector according to claim 1, wherein the 3′ terminus of said peptide sequence conferring resistance to an antibiotic is fused to the 5′ terminus of said SEQ ID NO: 1, 2 or 3.

9. The vector according to claim 1, wherein the fusion protein comprises SEQ ID NO: 23 or is encoded by SEQ ID NO: 22.

10. The vector according to claim 1, wherein said vector further comprises a nucleic acid sequence encoding a protein of interest.

11. The vector according to claim 10, wherein the nucleic acid sequence encoding said fusion protein and the nucleic acid sequence encoding said protein of interest (POI) are separated by an internal ribosomal entry site (IRES) sequence.

12. The vector according to claim 10, wherein said vector comprises:
a) one promoter or promoter assembly regulating the expression of both the fusion protein and the expression of the protein of interest (POI); or
b) at least two promoters, one regulating the expression of the fusion protein and the other one regulating the expression of said protein of interest (POI).

13. The vector according to claim 12, wherein said one or more promoters are promoters of the murine CMV immediate early region.

14. The vector according to claim 13, wherein said promoters are the IE1 and/or the IE2 promoters.

15. The vector according to claim 1, wherein said vector further comprises an amplification marker selected from the group consisting of adenosine deaminase (ADA), dihydrofolate reductase (DHFR), multiple drug resistance gene (MDR), ornithine decarboxylase (ODC) and N-(phosphonacetyl)-L-aspartate resistance (CAD).

16. A cell comprising a vector according to claim 1.

17. The cell according to claim 16, wherein said cell is selected from non-human mammalian cells or human cells.

18. A method of screening cells for expression of a protein of interest, said method comprising the steps of:
(i) transfecting cells with a vector according to claim 1;
(ii) selecting cell clones being resistant to an antibiotic selected from any of the antibiotics neomycin, kanamycin, neomycin-kanamycin, hygromycin, gentamycin, chloramphenicol, puromycin, zeocin or bleomycin;
(iii) incubating cells selected according to step (ii) with a solution containing a ligand with binding affinity to a sequence comprising SEQ ID NO: 1, 2 or 3 and fluorescent properties upon binding; and
(iv) detecting the fluorescence activity of cell clones selected according to step (ii) due to fluorescence of the ligand.

19. The method according to claim 18, wherein the ligand is:
a) a fluorescein derivative;
b) a membrane permeable biarsenic fluorescein derivative; or
c) 4′,5′-bis(1,3,2-dithioarsolan-2-yl)fluorescein.

20. The method according to claim 18, wherein said fluorescence activity is detected by fluorescence activated cell sorting (FACS) in step (iv).

21. The method according to claim 18, wherein the fluorescence activity of at least 20 cells are detected in step (iv).

22. The method according to claim 18, further comprising selecting about 5% to about 20% of the cells assayed in step (iv), wherein the selected cells are those exhibiting highest fluorescence activity in step (iv).

23. The method according to claim 18, further comprising assaying the expression level of the protein of interest in the selected cells.

24. A method of obtaining a cell line expressing a protein of interest, said method comprising the steps of:
(i) screening cells according to the method of claim 18;
(ii) selecting the cell(s) exhibiting the highest expression of said protein of interest; and
(iii) establishing a cell line from said cell.

25. A method of producing a protein of interest, said method comprising culturing a cell according to claim 16 under conditions which permit expression of said protein of interest.

26. The method according to claim 25, further comprising the step of purifying said protein of interest.

27. A method of producing a fusion protein, said method comprising culturing the cell according to claim 16 under conditions which permit expression of said fusion protein.

28. The method according to claim 27, further comprising the step of purifying said fusion protein.


SEQUENCE LISTING (25 sequences)

SEQ ID NO: 1 - protein, 6 aa, Artificial
  Generic binding sequence of a ligand having fluorescent properties
CCXXCC

SEQ ID NO: 2 - protein, 6 aa, Artificial
  More specific binding sequence of a ligand having fluorescent properties
CCXXCC

SEQ ID NO: 3 - protein, 10 aa, Artificial
  Binding sequence of a ligand having fluorescent properties, N- and C- terminally extended
GCCPGCCGGG

SEQ ID NO: 4 - DNA, 600 bp, Artificial
  Resistance gene for puromycin N-acetyltransferase derived from Streptomyces alboniger
atgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 5 - protein, 199 aa, Artificial
  Puromycin N-acetyltransferase derived from Streptomyces alboniger
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 6 - DNA, 795 bp, Artificial
  Neomycin resistance gene (encoding Neomycin phosphotransferase type II)
atggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttag

SEQ ID NO: 7 - protein, 264 aa, Artificial
  Neomycin phosphotransferase type II
MAKMRISPELKKLIEKYRCVKDTEGMSPAKVYKLVGENENLYLKMTDSRYKGTTYDVEREKDMMLWLEGKLPVPKVLHFERHDGWSNLLMSEADGVLCSEEYEDEQSPEKIIELYAECIRLFHSIDISDCPYTNSLDSRLAELDYLLNNDLADVDCENWEEDTPFKDPRELYDFLKTEKPEEELVFSHGDLGDSNIFVKDGKVSGFIDLGRSGRADKWYDIAFCVRSIREDIGEEQYVELFFDLLGIKPDWEKIKYYILLDELF

SEQ ID NO: 8 - DNA, 795 bp, Artificial
  Kanamycin resistance gene (encoding kanamycin phosphotransferase type II)
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 9 - protein, 264 aa, Artificial [sequence omitted]

SEQ ID NO: 10 - DNA, 810 bp, Artificial
  Neomycin-kanamycin resistance gene (encoding neomycin-kanamycin phosphotransferase type II)
ctgcagaccatgattgagcaggatggactgcatgctggcagccctgctgcttgggtggagagactgtttggctacgattgggctcagcagaccattggctgttctgatgccgctgtgttccggttgtctgcacaaggtcggcctgtgttgtttgtgaagacagatctgtctggagccctcaacgaactccaggatgaggctgccagactgagctggttggccaccacaggagtgccttgtgctgctgtgctggatgtggtgactgaggctggcagagactggctgctgctgggagaggtgcctggccaggacctgctgagcagccacctggccccagctgagaaagtcagcatcatggctgatgccatgagaagactgcacaccctggaccctgccacctgtcctttcgaccaccaagccaagcacagaattgagagagccagaaccagaatggaggctggcctggtggaccaggatgacctcgatgaggagcaccagggcctggccccagccgagttgtttgcccggttgaaggccagaatgccagatggagaggatttggtggtgacacatggagatgcctgtctgcctaacatcatggtggagaatggcagattctctggcttcattgactgtggccggctgggagtggctgacagataccaggacattgcattggccaccagagacattgcagaggagttgggaggagagtgggctgacagattcctggtgctgtatggcatcgctgcccctgacagccagagaatcgccttctaccggctgctggatgagttcttctgagagctc

SEQ ID NO: 11 - protein, 264 aa, Artificial [sequence omitted]

SEQ ID NO: 12 - DNA, 1026 bp, Artificial
  Hygromycin resistance gene (encoding hygromycin resistance protein)
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 13 - protein, 341 aa, Artificial
  Hygromycin resistance protein
MKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 14 - DNA, 534 bp, Artificial
  Gentamycin resistance gene (encoding gentamycin acetyl transferase)
atgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaa

SEQ ID NO: 15 - protein, 177 aa, Artificial
  Gentamycin acetyl transferase
MLRSSNDVTQQGSRPKTKLGGSSMGIIRTCRLGPDQVKSMRAALDLFGREFGDVATYSQHQPDSDYLGNLLRSKTFIALAAFDQEAVVGALAAYVLPRFEQPRSEIYIYDLAVSGEHRRQGIATALINLLKHEANALGAYVIYVQADYGDDPAVALYTKLGIREEVMHFDIDPSTAT

SEQ ID NO: 16 - DNA, 459 bp, Artificial
  Chloramphenicol resistance gene (encoding chloramphenicol acetyltransferase)
atgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaaatgctttttctctttctattattccatggacttcatttactgggtttaa

SEQ ID NO: 17 - protein, 152 aa, Artificial
  Chloramphenicol acetyltransferase
MNFNKIDLDNWKRKEIFNHYLNQQTTFSITTEIDISVLYRNIKQEGYKFYPAFIFLVTRVINSNTAFRTGYNSDGELGYWDKLEPLYTIFDGVSKTFSGIWTPVKNDFKEFYDLYLSDVEKYNGSGKLFPKTPIPEKCFFSFYYSMDFIYWV

SEQ ID NO: 18 - DNA, 375 bp, Artificial
  Zeomycin resistance gene
atggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 19 - protein, 124 aa, Artificial
  Zeomycin resistance protein
MAKLTSAVPVLTARDVAGAVEFWTDRLGFSRDFVEDDFAGVVRDDVTLFISAVQDQVVPDNTLAWVWVRGLDELYAEWSEVVSTNFRDASGPAMTEIGEQPWGREFALRDPAGNCVHFVAEEQD

SEQ ID NO: 20 - DNA, 435 bp, Artificial
  bleomycin resistance gene
atgaaaccaagaattagcatgattactctcggggttaaagaccttgaaaagtcagtcgtattttatcgtgatggcttaggatttcctcaaaaagaatcgccaccatcagtcgccttttttactctgaatggtacatggttaggtttgtatgatcgtgatgccttagcagaagatgctcaagtggctcctagtgataatactgagcaatctttttctggttttgcacttgcgcataatgtgaagtctgaaacggaagtcgatcaagtgttgactgaagtagaagccgcgggcgcgactgttaccaagcgcggtcagaaagtgttttggggtgggtattccggttacttcaaagatcttgatggctatttatgggaggttgcctataaccccttttgttggataggccctgaagattcgattgatataagatggtga

SEQ ID NO: 21 - protein, 144 aa, Artificial
  bleomycin resistance protein
MKPRISMITLGVKDLEKSVVFYRDGLGFPQKESPPSVAFFTLNGTWLGLYDRDALAEDAQVAPSDNTEQSFSGFALAHNVKSETEVDQVLTEVEAAGATVTKRGQKVFWGGYSGYFKDLDGYLWEVAYNPFCWIGPEDSIDIRW

SEQ ID NO: 22 - DNA, 630 bp, Artificial
  sequence encoding an exemplary inventive fusion protein, comprising as a first component a puromycin N-acetyltransferase sequence and as a second component a sequence according to SEQ ID NO 1 to 3
atgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggctgttgtcctggctgttgcggtggcggctag

SEQ ID NO: 23 - protein, 209 aa, Artificial
  Synthetic Construct
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAGCCPGCCGGG

SEQ ID NO: 24 - DNA, 22 bp, Artificial
  Primer oSerono1206
gtggctgcttatggtgacaatc

SEQ ID NO: 25 - DNA, 67 bp, Artificial
  Primer oSerono1239
cgcgctagctcattactagccgccaccgcaacagccaggacaacagccggcaccgggcttgcgggtc